SEQUENCE LISTING (34 sequences)

SEQ ID NO: 1 - DNA, 20 bp, Artificial Sequence
  Description of Artificial Sequence Synthetic oligonucleotide
ctaacagttgcttttatcac

SEQ ID NO: 2 - DNA, 7 bp, Artificial Sequence
  Description of Artificial Sequence Synthetic oligonucleotide
wgataar

SEQ ID NO: 3 - RNA, 20 nt, Artificial Sequence
  Description of Artificial Sequence Synthetic oligonucleotide
cuaacaguugcuuuuaucac

SEQ ID NO: 4 - protein, 9 aa, Unknown
  Description of Unknown “LAGLIDADG” family peptide motif sequence
LAGLIDADG

SEQ ID NO: 5 - DNA, 23 bp, Artificial Sequence
  Description of Artificial Sequence Synthetic oligonucleotide
  modified_base (21)..(23): a, c, t, g, unknown or other
ctaacagttgcttttatcacnnn

SEQ ID NO: 6 - DNA, 23 bp, Homo sapiens
ttaacagctgcctttatcactgc

SEQ ID NO: 7 - DNA, 23 bp, Homo sapiens
ttaacagctgcctttatcactgg

SEQ ID NO: 8 - DNA, 23 bp, Homo sapiens
cctgtgataaaagcaactgttag

SEQ ID NO: 9 - DNA, 23 bp, Homo sapiens
gcagtgataaaggcagctgttaa

SEQ ID NO: 10 - DNA, 23 bp, Homo sapiens
ctaacagttgcttttatcacagg

SEQ ID NO: 11 - DNA, 30 bp, Artificial Sequence
  Description of Artificial Sequence Synthetic oligonucleotide
agtttaacagctgcctttatcacgactacc

SEQ ID NO: 12 - DNA, 22 bp, Homo sapiens
atacagcttattttatcaccag

SEQ ID NO: 13 - DNA, 22 bp, Homo sapiens
atacagcttattttatcaccgg

SEQ ID NO: 14 - DNA, 28 bp, Homo sapiens
tcctggagcctgtgcactgccactcacg

SEQ ID NO: 15 - DNA, 28 bp, Homo sapiens
tcctggagcctgtgcactggcactcacg

SEQ ID NO: 16 - DNA, 28 bp, Artificial Sequence
  Description of Artificial Sequence Synthetic oligonucleotide
tcctggagcctgtgtcactggcactcac

SEQ ID NO: 17 - DNA, 21 bp, Artificial Sequence
  Description of Artificial Sequence Synthetic oligonucleotide
tcctggagcctggcactcacg

SEQ ID NO: 18 - DNA, 28 bp, Artificial Sequence
  Description of Artificial Sequence Synthetic oligonucleotide
tcctggagcctgtgacactggcactcac

SEQ ID NO: 19 - DNA, 28 bp, Artificial Sequence
  Description of Artificial Sequence Synthetic oligonucleotide
tcctggagcctgtgatcactggcactca

SEQ ID NO: 20 - DNA, 23 bp, Artificial Sequence
  Description of Artificial Sequence Synthetic oligonucleotide
tcctggagcctgtggcactcacg

SEQ ID NO: 21 - DNA, 15 bp, Artificial Sequence
  Description of Artificial Sequence Synthetic oligonucleotide
tcctggagcctcacg

SEQ ID NO: 22 - DNA, 28 bp, Artificial Sequence
  Description of Artificial Sequence Synthetic oligonucleotide
tcctggagcctgtgattcactggcactc

SEQ ID NO: 23 - DNA, 40 bp, Homo sapiens
agtttaacagctgcctttatcactgccactcacgactacc

SEQ ID NO: 24 - DNA, 40 bp, Homo sapiens
agtttaacagctgcctttatcactggcactcacgactacc

SEQ ID NO: 25 - DNA, 40 bp, Artificial Sequence
  Description of Artificial Sequence Synthetic oligonucleotide
agtttaacagctgcctttattcactggcactcacgactac

SEQ ID NO: 26 - DNA, 28 bp, Artificial Sequence
  Description of Artificial Sequence Synthetic oligonucleotide
agtttaacagctggcactcacgactacc

SEQ ID NO: 27 - DNA, 27 bp, Artificial Sequence
  Description of Artificial Sequence Synthetic oligonucleotide
agtttaacactggcactcacgactacc

SEQ ID NO: 28 - DNA, 37 bp, Artificial Sequence
  Description of Artificial Sequence Synthetic oligonucleotide
agtttaacagctgcctttactggcactcacgactacc

SEQ ID NO: 29 - DNA, 38 bp, Artificial Sequence
  Description of Artificial Sequence Synthetic oligonucleotide
agtttaacagctgcctttatctggcactcacgactacc

SEQ ID NO: 30 - DNA, 39 bp, Artificial Sequence
  Description of Artificial Sequence Synthetic oligonucleotide
agtttaacagctgcctttatactggcactcacgactacc

SEQ ID NO: 31 - DNA, 35 bp, Artificial Sequence
  Description of Artificial Sequence Synthetic oligonucleotide
agtttaacagctgccttttaacactcacgactacc

SEQ ID NO: 32 - DNA, 27 bp, Artificial Sequence
  Description of Artificial Sequence Synthetic oligonucleotide
agtttaacagctgcactcacgactacc

SEQ ID NO: 33 - DNA, 25 bp, Artificial Sequence
  Description of Artificial Sequence Synthetic oligonucleotide
agtttaacagctgctcacgactacc

SEQ ID NO: 34 - DNA, 39 bp, Artificial Sequence
  Description of Artificial Sequence Synthetic oligonucleotide
agtttaacagctgcctttacactggcactcacgactacc

(19) United States
(12) Patent Application Publication
(10) Pub. No.: US 2024/0245725 A1
(43) Pub. Date: Jul. 25, 2024

(54) METHODS FOR STRATIFYING SUBJECTS FOR FETAL HEMOGLOBIN REINDUCTION

(71) Applicant: THE CHILDREN'S MEDICAL CENTER CORPORATION, Boston, MA (US)

(72) Inventor: Daniel E. BAUER, Cambridge, MA (US)

(73) Assignee: THE CHILDREN'S MEDICAL CENTER CORPORATION, Boston, MA (US)

(21) Appl. No.: 18/290,405

(22) PCT Filed: May 10, 2022
(86) PCT No.: PCT/US2022/028432

§ 371 Date: Nov. 13, 2023

Related U.S. Application Data
(60) Provisional application No. 63/188,176, filed on May 13, 2021.

(51) Int. Cl. A61K 35/28; A61K 48/00; C12N 5/0789; C12N 9/22; C12N 15/11; C12Q 1/6883

(52) U.S. Cl. CPC A61K 35/28; A61K 48/005; C12N 5/0647; C12N 9/22; C12N 15/111; C12Q 1/6883; C12N 2310/20

(57) ABSTRACT

Provided herein are methods and compositions for improving safety and efficacy of hemoglobinopathy treatment by assessing the presence or absence of a given SNP polymorphism that affects CRISPR/Cas mediated reinduction of fetal hemoglobin (i.e., β-globin) in a cell by disrupting BCL11A expression at the genomic level.

### CROSS-REFERENCE TO RELATED APPLICATIONS

This application is a 35 U.S.C. § 371 National Phase Entry Application of International Application No. PCT/US2022/028432 filed May 10, 2022, which designated the U.S., which claims benefit under 35 U.S.C. § 119(e) to U.S. Provisional Application No. 63/188,176 filed May 13, 2021, the contents of each of which are incorporated herein by reference in their entirety.

### GOVERNMENT SUPPORT

This invention was made with government support under grant number HL154984, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 10, 2022, is named 701039-190060WOPT_SL.txt and is 7,371 bytes in size.

### TECHNICAL FIELD

The field of the invention relates to methods of selecting subjects for a given treatment of hemoglobinopathies by increasing expression of fetal hemoglobin.

### BACKGROUND

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickle) hemoglobin (HbS). HbS is prone to polymerization, particularly under deoxygenated conditions. HbS RBCs are more fragile than normal RBCs and undergo hemolysis more readily, leading eventually to anemia.

Recently, the search for treatment aimed at reduction of globin chain imbalance or predisposition to hemoglobin polymerization in patients with β-hemoglobinopathies has focused on the pharmacologic manipulation of fetal hemoglobin (α2γ2; HbF) or genomic manipulation of genes that are involved in expression of fetal hemoglobin (e.g., BCL11a).

### SUMMARY

The methods and compositions described herein are based, in part, on the discovery of a SNP polymorphism in a subset of subjects that causes off-target recognition and effects by the BCL11a guide RNA #1617 (sgRNA 1617) in a therapeutic CRISPR/Cas mediated system used to induce fetal hemoglobin expression. Subjects lacking this SNP polymorphism do not experience such off-target effects and can be safely treated with a CRISPR-Cas system using BCL11a sgRNA 1617, while those having the SNP polymorphism should not be administered this treatment or selecting a high-fidelity CRISPR/Cas system that reduces this off-target risk.

Thus, provided herein are methods and compositions for improving treatment of hemoglobinopathies by assessing the presence or absence of a given SNP polymorphism that affects CRISPR/Cas mediated reinduction of fetal hemoglobin (i.e., β-globin) in a cell by disrupting BCL11A expression at the genomic level.

One aspect provided herein relates to a method for enhancing levels of a fetal hemoglobin isoform in a subject in need thereof, the method comprising: (i) receiving results of a SNP polymorphism assay from a biological sample obtained from a subject indicating the absence of a SNP polymorphism at site rs114518452, (ii) modifying the genome of at least one hematopoietic stem cell to inhibit BCL11A, and (iii) administering the modified hematopoietic stem cell of step (ii) to the subject in need thereof, thereby enhancing levels of a fetal hemoglobin isoform in the subject.

Another aspect provided herein relates to a method for treating a hemoglobinopathy in a subject, the method comprising: (i) receiving results of a SNP polymorphism assay from a biological sample obtained from a subject indicating the absence of a SNP polymorphism at site rs114518452, (ii) modifying the genome of at least one hematopoietic stem cell to inhibit BCL11A, and (iii) administering the modified hematopoietic stem cell of step (ii) to a subject having a hemoglobinopathy, thereby treating the hemoglobinopathy in the subject.

In one embodiment of this aspect and all other aspects provided herein, the fetal hemoglobin isoform comprises a γ subunit.

In another embodiment of this aspect and all other aspects provided herein, the subject in need thereof comprises a hemoglobinopathy.

In another embodiment of this aspect and all other aspects provided herein, the hemoglobinopathy comprises a sickle cell disease.

In another embodiment of this aspect and all other aspects provided herein, the sickle cell disease is sickle cell anemia, sickle-hemoglobin C disease (HbSC), sickle beta-plus-thalassaemia (HbS/β+), or sickle beta-zero-thalassaemia (HbS/β0).

In another embodiment of this aspect and all other aspects provided herein, the hemoglobinopathy is a β-thalassemia.

In another embodiment of this aspect and all other aspects provided herein, the method further comprises a step of analyzing the subject's genome for a SNP polymorphism at site rs114518452.

In another embodiment of this aspect and all other aspects provided herein, the step of modifying the genome comprises contacting the hematopoietic stem cell with a CRISPR/Cas genome modification system and a BCL11A specific guide RNA.

In another embodiment of this aspect and all other aspects provided herein, the BCL11A specific guide RNA comprises BCL11A sgRNA 1617 targeting a protospacer sequence of: CTAACAGTTGCTITATCAC (SEQ ID NO: 1), and in some embodiments, possessing a spacer sequence of: CUAACAGUUGCUUUUAUCAC (SEQ ID NO: 3).

In another embodiment of this aspect and all other aspects provided herein, the hematopoietic stem cell is isolated from the subject and step (ii) is performed ex vivo.

In another embodiment of this aspect and all other aspects provided herein, the hematopoietic stem cell is isolated from a donor and step (ii) is performed ex vivo.

In another embodiment of this aspect and all other aspects provided herein, the hematopoietic stem cell is derived from an induced pluripotent stem cell or an embryonic stem cell.

In another embodiment of this aspect and all other aspects provided herein, the base at site rs114518452 is G (i.e., lacking the SNP polymorphism).

Another aspect provided herein relates to a method for selecting a guide RNA for a CRISPR/Cas based hemoglobinopathy treatment for a subject in need thereof, the method comprising: (i) receiving results of a SNP polymorphism assay from a biological sample obtained from a subject indicating the absence of a SNP polymorphism at site rs114518452, (ii) selecting BCL11A sgRNA 1617 as a guide RNA for use with a CRISPR/Cas based hemoglobinopathy treatment.

In one embodiment of this aspect and all other aspects provided herein, the nucleotide base at site rs114518452 is G (i.e., lacking the SNP polymorphism).

In another embodiment of this aspect and all other aspects provided herein, the method further comprises a step of analyzing the subject's genome for a SNP polymorphism at site rs114518452.

Another aspect provided herein relates to a method for selecting a guide RNA for a CRISPR/Cas based hemoglobinopathy treatment for a subject in need thereof, the method comprising: (i) receiving results of a SNP polymorphism assay from a biological sample obtained from a subject indicating the presence of a SNP polymorphism at site rs114518452, (ii) excluding BCL11A sgRNA 1617 as a guide RNA for use with a CRISPR/Cas based hemoglobinopathy treatment.

In one embodiment of this aspect and all other aspects described herein, the nucleotide base at site rs114518452 is C.

In another embodiment of this aspect and all other aspects provided herein, the method further comprises a step of analyzing the subject's genome for a SNP polymorphism at site rs114518452.

Another aspect provided herein relates to a method for optimizing a CRISPR/Cas based hemoglobinopathy treatment for a subject in need thereof, the method comprising: receiving results of a SNP polymorphism assay from a biological sample obtained from a subject indicating the presence of a SNP polymorphism at site rs114518452 and (a) excluding BCL11A sgRNA 1617 as a guide RNA for use with a CRISPR/Cas based hemoglobinopathy treatment, or (b) selecting BCL11A sgRNA 1617 as a guide RNA for use with a high-fidelity Cas enzyme.

In one embodiment of this aspect and all other aspects provided herein, the high-fidelity Cas enzyme comprises Cas9 R691A.

Another aspect provided herein relates to a hematopoietic stem cell composition comprising a hematopoietic stem cell modified to inhibit BCL11A for use in a method of treating a subject with a hemoglobinopathy, wherein the method comprises receiving results of a SNP polymorphism assay from a biological sample obtained from the subject indicating the absence of a SNP polymorphism at site rs114518452.

### BRIEF DESCRIPTION OF DRAWINGS

FIGS. 1A-1F CRISPRme: web-based analysis of off-target potential of CRISPR-Cas gene editing reflecting population genetic diversity. FIG. 1A CRISPRme software takes as input Cas protein type, protospacer and PAM sequence, reference genome, variants, homology threshold and genomic annotations and provides comprehensive, target-focused and individual-focused analyses of off-target potential. It is available as an online webtool and it can be deployed locally or used offline as command-line software. FIG. 1A discloses SEQ ID NO: 1. FIG. 1B Analysis of BCL11A-1617 spacer targeting the +58 erythroid enhancer with SpCas9, NNN PAM, 1000 Genome Project variants, mismatches up to 6 and bulges up to 2. FIG. 1B discloses SEQ ID NO: 5. FIG. 1C The off-target site with the highest CFD score is created by the minor allele of rs114518452. FIG. 1C discloses SEQ ID NOS 5, 6 and 7, respectively, in order of appearance. FIG. 1D Top 1000 off-target sites ranked by CFD score, indicating the CFD score of the reference and where applicable alternative allele, with allele frequency indicated by circle size. FIG. 1E The top predicted off-target site from CRISPRme is an allele-specific target with 3 mismatches to the BCL11A-1617 spacer sequence and where rs114518452-C minor allele produces a de novo NGG PAM sequence. PAM sequence shown in bold and mismatches to BCL11A-1617 shown as lowercase. Coordinates are hg38. FIG. 1E discloses SEQ ID NOS 8, 9, 10, and 6, respectively, in order of appearance (left to right, top to bottom). FIG. 1F rs114518452 allele frequencies based on gnomAD v3.1.

FIGS. 2A-2E CRISPRme provides analysis of off-target potential of CRISPR-Cas gene editing reflecting private genetic diversity. FIG. 2A CRISPRme analysis was conducted with variants from HGDP comprising WGS from 929 individuals of diverse ancestry. Variant off-targets with greater CFD score from HGDP variants as compared to reference genome or 1000G variants are plotted, sorted by CFD score, with HGDP variants shown and reference or 1000G variants shown in red (reference). FIG. 2B HGDP variant off-targets with CFD≥0.2 and the increase in CFD due to HGDP variants ≥0.1. Individual samples from each of the seven super-populations were shuffled 100 times to calculate mean and 95% confidence interval. FIG. 2C Intersection analysis of HGDP variant off-targets with CFD≥0.2 and the increase in CFD due to HGDP variant ≥0.1. Shared variants were found in 2 or more samples and private variants limited to a single sample. FIG. 2D CRISPRme analysis of a single individual (HGDP01211) showing top 100 variant off-targets from each of the following three categories: CFD score shared with 1000G variants (left panel), higher CFD score compared to reference genome or 1000G variants but shared with other HGDP samples (center panel), and higher CFD score compared to reference genome or 1000G variants and not found in any other sample (right panel). For center and right panels, reference refers to CFD score from reference genome or 1000G variants. FIG. 2E The top predicted private off-target site from HGDP01211 is an allele-specific target with 3 mismatches to the BCL11A-1617 spacer sequence and 1 RNA bulge where rs1191022522-G minor allele produces a NGG PAM sequence. FIG. 2E discloses SEQ ID NOS 5, 12, and 13, respectively, in order of appearance.

FIGS. 3A-3D. Allele-specific off-target editing due to an African-ancestry specific variant by a BCL11A enhancer targeting gRNA. FIG. 3A Human CD34+ HSPCs from a donor heterozygous for rs114518452-G/C (Donor 1, REF/ALT) were subject to 3×NLS-SpCas9:sg1617 RNP electroporation followed by amplicon sequencing of the off-target site around chr2:210,530,659-210,530,681 (off-target-rs114518452). Spacer mismatches shown by lowercase. Coordinates hg38. Corresponding CFD scores for reference and alternative alleles indicated and representative aligned reads shown. The rs114518452 position is shown in bold. FIG. 3A discloses SEQ ID NOS 1, 23, 24, 23, 24, 25, 26, 27, 28, 29, 30, 31, 32, 33, 34, and 11, respectively, in order of appearance. FIG. 3B Reads classified based on allele (indeterminate if the rs114518452 position is deleted) and presence or absence of indels (edits). FIG. 3C Human CD34+ HSPCs from a donor heterozygous for rs114518452-G/C (Donor 1) were subject to 3×NLS-SpCas9:sg1617 RNP electroporation, HiFi-3×NLS-SpCas9:sg1617 RNP electroporation, or no electroporation (mock) followed by amplicon sequencing of the on-target and off-target-rs114518452 sites. Each dot represents an independent biological replicate (n=3). Indel frequency was quantified for reads aligning to either the reference or alternative allele. FIG. 3D Human CD34+ HSPCs from 6 donors homozygous for rs114518452-G/G (Donors 2-7, REF/REF) were subject to 3×NLS-SpCas9:sg1617 RNP electroporation followed by amplicon sequencing of the on-target and OT-rs114518452 sites.

FIG. 4A-4D. Allele-specific pericentric inversion following BCL11A enhancer editing. FIG. 4A Concurrent cleavage of the on-target and off-target-rs114518452 sites could lead to pericentric inversion of chr2 as depicted. PCR primers F1, R1, F2, and R2 were designed to detect potential inversions. FIG. 4B Human CD34+ HSPCs from a donor heterozygous for rs114518452-G/C (Donor 1) were subject to 3×NLS-SpCas9:sg1617 RNP electroporation, HiFi-3×NLS-SpCas9:sg1617 RNP electroporation, or no electroporation with 3 biological replicates. Human CD34+ HSPCs from 6 donors homozygous for rs114518452-G/G (Donors 2-7, REF/REF) were subject to 3×NLS-SpCas9:sg1617 RNP electroporation with 1 biological replicate per donor. Gel electrophoresis for inversion PCR performed with F1/F2 and R1/R2 primer pairs on left and right respectively with expected sizes of precise inversion PCR products indicated. FIG. 4C Reads from amplicon sequencing of the F1/F2 product (expected to include the rs114518452 position) from 3×NLS-SpCas9:sg1617 RNP treatment were aligned to reference and alternative inversion templates. The rs114518452 position is shown in bold. FIG. 4C discloses SEQ ID NOS 14, 15, 16, 17, 18, 19, 15, 20, 21, and 22, respectively, in order of appearance. FIG. 4D Reads classified based on allele (indeterminate if the rs114518452 position deleted).

### DETAILED DESCRIPTION

Provided herein are methods and compositions for ensuring safe and efficacious treatment of hemoglobinopathies by assessing the presence or absence of a given SNP polymorphism that affects CRISPR/Cas mediated reinduction of fetal hemoglobin (i.e., β-globin) in a cell by disrupting BCL11A expression at the genomic level. The SNP polymorphism rs114518452 occurs at position chr2:210530659 (GRCh38/g38) and comprises a G to C switch on the 5′ to 3′ strand (i.e., the presence of the rs114518452 polymorphism is comprised by a cytosine at position chr2:210530659 (GRCh38/g38) on the 5′ to 3′ strand while the absence of the polymorphism is comprised by a guanine on the 5′ to 3′ strand).

The BCL11A protein acts as a stage specific regulator of fetal hemoglobin expression by repressing γ-globin induction. Thus, inhibition of BCL11a activity or expression, particularly at the genomic level can induce fetal hemoglobin induction. Genomic modification methods for BCL11A are described in detail in US Patent Application Nos.: US2021-0047632, and US2021-0115420 and in U.S. Pat. No. 10,472,619, the contents of each of which are incorporated by reference herein in their entirety.

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the term “single nucleotide polymorphism (SNP)” refers to a variant at a single nucleotide site in the genome of a subject that can arise due to the substitution, deletion or insertion of a nucleotide residue at a polymorphic site. SNPs may occur in protein coding regions, in which case different polymorphic forms of the sequence may give rise to variant protein sequences.

As used herein, the term “SNP polymorphism at site rs114518452” refers to the presence of a cytosine (C) at site rs114518452, chr2:210530659 (GRCh38/hg38). Thus, a subject is determined to have the SNP polymorphism if the nucleotide at site rs114518452 is cytosine (C). The converse is also true; a subject is determined to lack the SNP polymorphism (i.e., wild-type) if the nucleotide at site rs114518452 is guanine (G). The term “rs114518452-G/C” refers to the SNP polymorphism G to C switch relative to the 5′ to 3′ strand of DNA.

As used herein, the term “high-fidelity DNA targeting endonuclease” refers to an endonuclease that, at a minimum, generates a double-stranded break at a desired position in the genome (e.g., chr2:60489054-60501477 (GRCh38/hg38) or more specifically at e.g., chr2:60,495,266-60,495,267 (GRCh38/hg38)) but does not generate a double-stranded break at the SNP polymorphism site rs114518452 (i.e., low or absent off-target frequency) when using BCL11A sgRNA 1617. In addition, a high-fidelity nuclease (e.g., high-fidelity Cas9) will greatly reduce the chance of off-target cleavage while only minimally reducing the on-target cleavage (e.g., increasing the specificity). The use of a high-fidelity nuclease need not eliminate the off-target frequency entirely, but the reduction can still be meaningful in terms of risk of genotoxicity. In some embodiments, the off-target frequency of a high-fidelity nuclease is reduced by at least 20%, at least 30%, at least 40%, at least 50%, at least 60%, at least 70%, at least 80%, at least 90%, at least 100% (i.e., off-target frequency below detectable limits) compared to the off-target frequency with a low-fidelity nuclease is used assuming all other reagents and conditions are equal.

As used herein, the term “off-target effect” refers to the generation of a double-stranded break at the SNP polymorphism site rs114518452-C when the BCL11A sgRNA 1617 is used in a CRISPR/Cas modification system.

As used herein the term “increasing fetal hemoglobin levels” in a cell indicates that protein expression of fetal hemoglobin is at least 5% higher in a subject following treatment with a hematopoietic stem or progenitor cell having a modified genome that inhibits BCL11A than in a comparable, control hematopoietic stem or progenitor cell lacking a modified genome. It is preferred that the percentage of fetal hemoglobin expression in a subject treated with a hematopoietic stem or progenitor cell having a modified genome that inhibits BCL11A is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a substantially similar, control hematopoietic stem or progenitor cell lacking genomic modification. The term “control treated population” is used herein to describe a population of hematopoietic stem or progenitor cells that has been treated with identical media, viral induction, nucleic acid sequences, temperature, confluency, flask size, pH, etc., with the exception of genomic modification of DNA (e.g., at chromosome 2 location 60,716,189-60,728,612, the GATA binding site of BCL11A, which inhibits BCL11A). In one embodiment, any method known in the art can be used to measure an increase in fetal hemoglobin expression, e. g. Western Blot analysis of fetal γ-globin protein and quantifying mRNA of fetal γ-globin.

The term “isolated population” with respect to an isolated population of cells (e.g., hematopoietic stem or progenitor cells) as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of human hematopoietic progenitor cells, e.g., a substantially pure population of human hematopoietic progenitor cells as compared to a heterogeneous population of cells comprising human hematopoietic progenitor cells and cells from which the human hematopoietic progenitor cells were derived.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of hematopoietic progenitor cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not hematopoietic progenitor cells as defined by the terms herein. It is contemplated herein that treatment as described herein does not require a substantially pure population and can instead comprise a hematopoietic stem or progenitor cell (e.g., having a genomic modification) in a heterogeneous mixture with other cell types or with hematopoietic stem or progenitor cells lacking such genomic modification.

As used herein, the term “treating” includes reducing or alleviating at least one adverse effect or symptom of a condition, disease or disorder, e.g., a hemoglobinopathy. For example, the term “treating” and “treatment” refers to administering to a subject an effective amount of a composition, e.g., an effective amount of a composition comprising a population of hematopoietic progenitor cells so that the subject has a reduction in at least one symptom of the disease (e.g., pain or sickle cell flare) or an improvement in the disease, for example, beneficial or desired clinical results. For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. Successful treatment can also be assessed by a reduction in the need for medical interventions (e.g., hydroxyurea), reduction in hospital or emergency room visits, reduction in anemia, or other markers of an improved quality of life. In some embodiments, treatment can include prophylaxis. However, in alternative embodiments, treatment does not include prophylaxis.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

As used herein, “prevention” or “preventing,” when used in reference to a disease, disorder or symptoms thereof, refers to a reduction in the likelihood that an individual will develop a disease or disorder or the likelihood that an individual will experience a “flare” of “pain crisis” of the disorder (i.e., becomes symptomatic), e.g., a hemoglobinopathy such as sickle cell disease. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention. In addition, the reduction of the number of flares, reduced need for pharmacologic or medical interventions, or improved quality of life are indicative of “preventing” the at least one symptom of a hemoglobinopathy.

As used herein, the term “subject” includes humans and mammals. The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. In some preferred embodiments, a mammal is a human. A subject can be of any age including a neonate toddler, child, teen, adult or a geriatric subject. Typically, a subject in need thereof will have negligible levels of fetal hemoglobin at the time of treatment. In some embodiments, the levels of fetal hemoglobin in a subject are assessed prior to treatment or as a method for monitoring effective treatment of a subject.

In one embodiment, the subject or mammal has been diagnosed with a hemoglobinopathy. In a further embodiment, the hemoglobinopathy is a β-hemoglobinopathy

As used herein, the term “hemoglobinopathy” means any defect in the structure or function of any hemoglobin of an individual, and includes defects in the primary, secondary, tertiary or quaternary structure of hemoglobin caused by any mutation, such as deletion mutations or substitution mutations in the coding regions of the β-globin gene, or mutations in, or deletions of, the promoters or enhancers of such genes that cause a reduction in the amount of hemoglobin produced as compared to a normal or standard condition. The term further includes any decrease in the amount or effectiveness of hemoglobin, whether normal or abnormal, caused by external factors such as disease, chemotherapy, toxins, poisons, or the like. Exemplary hemoglobinopathies include sickle cell disease or β-thalassemia. As used herein, the term “sickle cell disease” can be sickle cell anemia, sickle-hemoglobin C disease (HbSC), sickle beta-plus-thalassaemia (HbS/β+), or sickle beta-zero-thalassaemia (HbS/β0). In another preferred embodiment, the hemoglobinopathy is a β-thalassemia.

In one embodiment, the term “effective amount”, as used herein, refers to the amount of a cell composition comprising hematopoietic stem or progenitor cells comprising a modified genome that is safe and sufficient to treat, lesson the likelihood of, or delay the development of a hemoglobinopathy. The amount can thus cure or result in amelioration of the symptoms of the hemoglobinopathy, slow the course of hemoglobinopathy disease progression, slow or inhibit a symptom of a hemoglobinopathy, slow or inhibit the establishment of secondary symptoms of a hemoglobinopathy or inhibit the development of a secondary symptom of a hemoglobinopathy. The effective amount for the treatment of the hemoglobinopathy depends on the type of hemoglobinopathy to be treated, the severity of the symptoms, the subject being treated, the age and general condition of the subject, the mode of administration and so forth. Thus, it is not possible or prudent to specify an exact “effective amount”. However, for any given case, an appropriate “effective amount” can be determined by one of ordinary skill in the art using only routine experimentation.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

### Hemoglobinopathies

Fetal hemoglobin (HbF) is a tetramer of two adult α-globin polypeptides and two fetal β-like γ-globin polypeptides. During gestation, the duplicated γ-globin genes constitute the predominant genes transcribed from the β-globin locus. Following birth, γ-globin becomes progressively replaced by adult pi-globin, a process referred to as the “fetal switch” (3). The molecular mechanisms underlying this switch have remained largely undefined and have been a subject of intense research. The developmental switch from production of predominantly fetal hemoglobin or HbF (α₂γ₂) to production of adult hemoglobin or HbA (α₂β₂) begins at about 28 to 34 weeks of gestation and continues shortly after birth at which point HbA becomes predominant. This switch results primarily from decreased transcription of the gamma-globin genes and increased transcription of beta-globin genes. On average, the blood of a normal adult contains only about 2% HbF, though residual HbF levels have a variance of over 20 fold in healthy adults (Atweh, Semin. Hematol. 38(4):367-73 (2001)).

Hemoglobinopathies encompass a number of anemias of genetic origin in which there is a decreased production and/or increased destruction (hemolysis) of red blood cells (RBCs). These disorders also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders specifically associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)). Moreover, the presence of a BCL11A genetic variant, HBS1L-MYB variation, ameliorates the clinical severity in beta-thalassemia. This variant has been shown to be associated with HbF levels. It has been shown that there is an odds ratio of 5 for having a less severe form of beta-thalassemia with the high-HbF variant (Galanello S. et al., 2009, Blood, in press).

The search for treatment aimed at reduction of globin chain imbalance in patients with β-hemoglobinopathies has focused on the pharmacologic manipulation of fetal hemoglobin (α2γ2; HbF). The important therapeutic potential of such approaches is suggested by observations of the mild phenotype of individuals with co-inheritance of both homozygous β-thalassemia and hereditary persistence of fetal hemoglobin (HPFH), as well as by those patients with homozygous β-thalassemia who synthesize no adult hemoglobin, but in whom a reduced requirement for transfusions is observed in the presence of increased concentrations of fetal hemoglobin. Furthermore, it has been observed that certain populations of adult patients with β chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease (Pembrey, et al., Br. J. Haematol. 40: 415-429 (1978)). It is now accepted that O-hemoglobinopathies, such as sickle cell anemia and the β-thalassemias, are ameliorated by increased HbF production. (Reviewed in Jane and Cunningham Br. J. Haematol. 102: 415-422 (1998) and Bunn, N. Engl. J. Med. 328: 129-131 (1993)).

There is accumulating evidence that external factors can influence γ-globin gene expression. The first group of compounds discovered having HbF reactivation activity were cytotoxic drugs. The ability to cause de novo synthesis of HbF by pharmacological manipulation was first shown using 5-azacytidine in experimental animals (DeSimone, Proc Natl Acad Sci USA. 79(14):4428-31 (1982)). Subsequent studies confirmed the ability of 5-azacytidine to increase HbF in patients with β-thalassemia and sickle cell disease (Ley, et al., N. Engl. J. Medicine, 307: 1469-1475 (1982), and Ley, et al., Blood 62: 370-380 (1983)). Additional experiments demonstrated that baboons treated with cytotoxic doses of arabinosylcytosine (ara-C) responded with striking elevations of F-reticulocytes (Papayannopoulou et al., Science. 224(4649):617-9 (1984)), and that treatment with hydroxyurea led to induction of γ-globin in monkeys or baboons (Letvin et. al., N Engl J Med. 310(14):869-73 (1984)).

The second group of compounds investigated for the ability to cause HbF reactivation activity was short chain fatty acids. The initial observation in fetal cord blood progenitor cells led to the discovery that γ-aminobutyric acid can act as a fetal hemoglobin inducer (Perrine et al., Biochem Biophys Res Commun. 148(2):694-700 (1987)). Subsequent studies showed that butyrate stimulated globin production in adult baboons (Constantoulakis et al., Blood. December; 72(6):1961-7 (1988)), and it induced γ-globin in erythroid progenitors in adult animals or patients with sickle cell anemia (Perrine et al., Blood. 74(1):454-9 (1989)). Derivatives of short chain fatty acids such as phenylbutyrate (Dover et al., Br J Haematol. 88(3):555-61 (1994)) and valproic acid (Liakopoulou et al., 1: Blood. 186(8):3227-35 (1995)) also have been shown to induce HbF in vivo. Given the large number of short chain fatty acid analogs or derivatives of this family, there are a number of potential compounds of this family more potent than butyrate. Phenylacetic and phenylalkyl acids (Torkelson et al., Blood Cells Mol Dis. 22(2):150-8. (1996)), which were discovered during subsequent studies, were considered potential HbF inducers as they belonged to this family of compounds. Presently, however, the use of butyrate or its analogs in sickle cell anemia and β-thalassemia remains experimental and cannot be recommended for treatment outside of clinical trials.

Clinical trials aimed at reactivation of fetal hemoglobin synthesis in sickle cell anemia and β-thalassemia have included short term and long term administration of such compounds as 5-azacytidine, hydroxyurea, recombinant human erythropoietin, and butyric acid analogs, as well as combinations of these agents. Following these studies, hydroxyurea was used for induction of HbF in humans and later became the first and only drug approved by the Food and Drug Administration (FDA) for the treatment of hemoglobinopathies. However, varying drawbacks have contraindicated the long term use of such agents or therapies, including unwanted side effects and variability in patient responses. For example, while hydroxyurea stimulates HbF production and has been shown to clinically reduce sickling crisis, it is potentially limited by myelotoxicity and the risk of carcinogenesis. Potential long term carcinogenicity would also exist in 5-azacytidine-based therapies. Erythropoietin-based therapies have not proved consistent among a range of patient populations. The short half-lives of butyric acid in vivo have been viewed as a potential obstacle in adapting these compounds for use in therapeutic interventions. Furthermore, very high dosages of butyric acid are necessary for inducing γ-globin gene expression, requiring catheritization for continuous infusion of the compound. Moreover, these high dosages of butyric acid can be associated with neurotoxicity and multiorgan damage (Blau, et al., Blood 81: 529-537 (1993)). While even minimal increases in HbF levels are helpful in sickle cell disease, β-thalassemias require a much higher increase that is not reliably, or safely, achieved by any of the currently used agents (Olivieri, Seminars in Hematology 33: 24-42 (1996)).

### BCL11A Inhibition

BCL11a activity and/or expression has been shown to repress expression of fetal hemoglobin isoforms. Thus, inhibition of BCL11a removes this repression and permits fetal hemoglobin isoforms to be re-induced, for example, in an adult. Thus, in some embodiments, an inhibitor of BCL11a is used to treat subjects for treatment of a hemoglobinopathy.

In some embodiments, the inhibitor of BCL11A interferes with BCL11A's interaction with its binding partners. By “interferes with BCL11A interactions with BCL11A binding partners” is meant that the amount of interaction of BCL11A with the BCL11A binding partner is at least 5% lower in populations treated with a BCL11A inhibitor, than a comparable, control population, wherein no BCL11A inhibitor is present. It is preferred that the amount of interaction of BCL11A with the BCL11A binding partner in a BCL11A-inhibitor treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no BCL11A inhibitor is added. At a minimum, BCL11A interaction can be assayed by determining the amount of BCL11A binding to the BCL11A binding partner using techniques standard in the art, including, but not limited to, mass spectrometry, immunoprecipitation, or gel filtration assays. Alternatively, or in addition, BCL11A activity can be assayed by measuring fetal hemoglobin expression at the mRNA or protein level following treatment with a candidate BCL11A inhibitor.

In one embodiment, BCL11A activity is the interaction of BCL11A with its binding partners: GATA-1, FOG-1, components of the NuRD complex, matrin-3, MTA2 and RBBP7. Accordingly, any antibody or fragment thereof, small molecule, chemical or compound that can block this interaction is considered an inhibitor of BCL11A activity.

In other embodiments, the BCL11A inhibitor acts to decrease BCL11A expression and/or activity. By “decreased BCL11A expression” is meant that the amount of expression of BCL11A is at least 5% lower in a cell or cell population treated with a DNA-targeting endonuclease, than a comparable, control cell or cell population, wherein no agent that deletes the GATA 1 binding element is present. It is preferred that the percentage of BCL11A expression in a treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no agent that deletes the GATA1 binding element is added.

By “decreases BCL11A activity” is meant that the amount of functional activity of BCL11A is at least 5% lower in a cell or cell population treated with the methods described herein, than a comparable, control cell or population, wherein no agent that deletes the GATA1 binding element is present. It is preferred that the percentage of BCL11A activity in a treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no agent that deletes the GATA1 binding element is added. At a minimum, BCL11A activity can be assayed by determining the amount of BCL11A expression at the protein or mRNA levels, using techniques standard in the art. Alternatively, or in addition, BCL11A activity can be determined using a reporter construct, wherein the reporter construct is sensitive to BCL11A activity. The γ-globin locus sequence is recognizable by the nucleic acid-binding motif of the BCL11A construct.

### SNP Polymorphism and Stratification of Patients

The SNP polymorphism at site rs114518452 (i.e., cytosine (C); a G to C switch on the 5′ to 3′ strand) can be detected by any known method of genotyping or any method of identifying differences between nucleic acid sequences known to those of skill in the art. A number of suitable methods are available in the art and include, but are not limited to, direct probing, allele specific hybridization, PCR, Allele Specific Amplification (ASA) (WO93/22456), Allele Specific Hybridization, single base extension (U.S. Pat. No. 4,656,127), ARMS-PCR, Taqman (U.S. Pat. Nos. 4,683,202; 4,683,195; and 4,965,188), oligo ligation assays, single-strand conformational analysis ((SSCP) Orita et al PNAS 86 2766-2770 (1989)), Genetic Bit Analysis (WO 92/15712) and RFLP direct sequencing, mass-spectrometry (MALDI-TOF) and DNA arrays.

The SNP polymorphism at site rs114518452 is determined to be “present” if the nucleotide base at that site is a cytosine (C) nucleotide relative to the 5′ to 3′ strand of genomic DNA. Accordingly, the SNP polymorphism at site rs114518452 is determined to be “absent” if the nucleotide base at that site comprises a guanine (G) nucleotide relative to the 5′ to 3′ strand of genomic DNA.

The above described methods may require amplification of a DNA sample from the subject, and this can be done by techniques known in the art such as PCR (see PCR Technology: Principles and Applications for DNA Amplification (ed. H. A. Erlich, Freeman Press, N Y 1992; PCR Protocols: A Guide to methods and Applications (eds. Innis et al., Academic press, San Diego, Calif. 1990); Mattila et al., Nucleic Acids Res. 19 4967 (1991); Eckert et al., PCR Methods and Applications 117 (1991) and U.S. Pat. No. 4,683,202. Other suitable amplification methods include ligase chain reaction (LCR) (Wu et al., Genomics 4 560 (1989); Landegran et al., Science 241 1077 (1988)), transcription amplification (Kwoh et al., Proc Natl Acad Sci USA 86 1173 (1989)), self-sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively.

Detection of the presence (or absence) of the SNP polymorphism at site rs11451452 is preferably carried out on a biological sample removed from a subject. Any biological sample comprising cells containing nucleic acid is suitable for this purpose. Examples of suitable samples include whole blood, leukocytes, semen, saliva, tears, buccal, skin or hair. For analysis of cDNA, mRNA or protein, the sample should be from a tissue in which the sequence of interest is expressed. Blood is a readily accessible sample. In some embodiments, the methods described herein further include the steps of obtaining a sample from a subject, and preparing nucleic acid from the sample.

Provided herein are methods for stratifying subjects to a given hemoglobinopathy treatment based on the presence or absence of a SNP polymorphism at site rs114518452 (relative to the 5′ to 3′ genomic DNA strand). As shown in the working Examples herein, the presence of a cytosine at site rs114518452 creates a protospacer adjacent motif (PAM), which causes an off-target double-strand break when the guide RNA for a therapeutic CRISPR/Cas system is BCL11A sgRNA 1617 (SEQ ID NO: 1). This off-target cleavage site can induce indel formation at that site (e.g., insertions, deletions, etc.). Such off-target effects can reduce the efficiency of genomic modification at the preferred site and/or introduce unwanted side-effects during genomic modification therapy. Thus, subjects determined to have this polymorphism cannot be safely and/or effectively treated with the guide RNA BCL11A sgRNA 1617. Rather, upon detection of this polymorphism, a subject should be stratified to treatment with a different BCL11A guide RNA or with the use of alternative genomic modification methods (e.g., meganucleases, TALENs etc.). Such subjects can also be treated by pharmacological means such as hydroxyurea. As shown in the Examples section herein, the use of a high-fidelity Cas enzyme, such as Cas9 R691A.

Subjects lacking this polymorphism can be safely and effectively treated using the BCL11A sgRNA 1617 as part of a CRISPR/Cas modification system. Given the effectiveness of this treatment, it is a preferred method for treating such subjects.

The SNP polymorphism, rs114518452-G/C, appears to be most prevalent in African subjects, thus in one embodiment the subject to be treated using the methods described herein is an African or African-American subject.

### Hematopoietic Stem or Progenitor Cells

Treatment of hemoglobinopathies can be achieved using hematopoietic stem or progenitor cells that have been modified to inhibit BCL11A, thereby producing fetal hemoglobin isoforms.

In one embodiment, the hematopoietic stem or progenitor cell is contacted ex vivo or in vitro with a BCL11A inhibitor or comprises a genomic modification that inhibits BCL11A expression and/or activity. In a specific embodiment, the cell being contacted is a cell of the erythroid lineage. In one embodiment, the cell composition comprises cells having decreased BCL11A expression.

“Hematopoietic stem or progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiments, the hematopoietic stem or progenitor cell has at least one of the cell surface marker characteristic of hematopoietic progenitor cells: CD34+, CD59+, Thy1/CD90+, CD38lo/−, and C-kit/CD117+. Preferably, the hematopoietic progenitor cells have several of these markers.

In some embodiments, the hematopoietic progenitor cells of the erythroid lineage have the cell surface marker characteristic of the erythroid lineage: CD71 and Ter119.

In one embodiment of this aspect, and all other aspects, the isolated progenitor cell or isolated cell is a hematopoietic progenitor cell, a human cell, a CD34+, or an induced pluripotent stem cell.

In one embodiment of this aspect, and all other aspects, the hematopoietic progenitor is a cell of the erythroid lineage. Methods of isolating hematopoietic progenitor cell are well known in the art, e.g., by flow cytometric purification of CD34+ or CD133+ cells, microbeads conjugated with antibodies against CD34 or CD133, markers of hematopoietic progenitor cell. Commercial kits are also available, e.g., MACS® Technology CD34 MicroBead Kit, human, and CD34 MultiSort Kit, human, and STEMCELL™ Technology EasySep™ Mouse Hematopoietic Progenitor Cell Enrichment Kit.

In another embodiment of this aspect and all other aspects described herein, the hematopoietic progenitor cell, the isolated human cell, or isolated cell is contacted ex vivo or in vitro.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

### Genomic Modification of BCL11a to Induce Fetal Hemoglobin Expression

Genomic modification to inhibit the BCL11a gene has been previously shown to be an effective means for increasing expression of fetal isoforms of hemoglobin. In certain methods of such genomic modification, a CRISPR/Cas system is used to inhibit BCL11a and in some cases, a BCL11a guide RNA (1617; SEQ ID NO. 1) can be used.

BCL11A sgRNA 1617
(SEQ ID NO: 1)
CTAACAGTTGCTTTTATCAC

The inventors have found a SNP polymorphism in a subset of patients that binds this guide RNA (BCL11a sgRNA 1617) and can result in off-target genomic modification (e.g., insertions, deletions etc.). Such off-target modifications are undesirable and are associated with an increased risk of unsafe therapy when BCL11a sgRNA 1617 is used.

Thus, provided herein are assays and methods for determining the presence or absence of a SNP polymorphism that poses a risk for off-target effects using a BCL11a targeted genomic modification (i.e., BCL11a sgRNA 1617 as part of a CRISPR/Cas genomic modification system). In the absence of the polymorphism, subjects can be safely treated with BCL11a sgRNA 1617 in a CRISPR/Cas system. In the presence of the polymorphism, treatment is restricted to the use of high-fidelity Cas enzymes to prevent off-target binding and effects at the site of the polymorphism or modification of BCL11a using an alternative guide RNA. In some embodiments, the subject is treated with an alternative gene editing system, like TALEN/ZFN, base editor, Cas enzyme besides SpCas9, etc all of which can still target the BCL11A enhancer. Alternatively, subjects having the polymorphism can be treated using non-genome modification methods (e.g., pharmacological interventions such as hydroxyurea).

In subjects lacking the polymorphism as described herein, the subject can be treated using a method for producing a progenitor cell having decreased BCL11A mRNA or protein expression comprising contacting an isolated progenitor cell with an agent that deletes the GATA1 binding element in the functional core of the BCL11A enhancer +58kb by making precise cleavages near the targeted GATA1 binding element to delete it, thereby reducing the mRNA or protein expression of BCL11A.

As used herein, “precise cleavages near the targeted GATA1 binding element” refers to precise cleavages within at least 100 base pairs of the start of the GATA1 binding element. The precise cleavages can be with 100 base pairs upstream or downstream of the start or end of the GATA1 binding element, respectively. The precise cleavages can be within at least 95, at least 90, at least 85, at least 80, at least 75, at least 70, at least 65, at least 60, at least 55, at least 50, at least 45, at least 40, at least 35, at least 30, at least 25, at least 20, at least 15, at least 10, at least 5, at least 4, at least 3, at least 2, at least 1, or less base pairs of the start or end of the GATA1 binding element. If at least two precise cleavages are made, the at least two precise cleavages can all be present upstream of the GATA 1 binding element, downstream of the GATA1 binding element, or can be present both upstream and downstream of the GATA1 binding element.

Another aspect of treatment provided herein relates to an isolated genetic engineered human cell having at least one genetic modification on chromosome 2 location 60,716,189-60,728,612, (according to UCSC Genome Browser hg 19 human genome assembly) near the GATA1 binding element in the functional core of the BCL11A enhancer +58kb generated by any method described herein. In one embodiment, the decrease of BCL11A mRNA or protein expression is achieved by causing at least one genetic modification at the genomic DNA of the cell on chromosome 2 location 60,716,189-60,728,612 that results in epigenetic modification of the genetic function at chromosome 2 location 60,716,189-60,728,612. In this embodiment, the BCL11A enhancer activity located within this chromosome 2 location 60,716,189-60,728,612 is reduced. By decrease in this aspect, the enhancer activity in enhancing BCL11A mRNA or protein expression in the cell is at least 5% lower, at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that is not treated with any method disclosed herein. By a “decrease of the BCL11A mRNA or protein expression in the cell” is meant that protein expression is at least 5% lower, at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that is not treated in any method disclosed herein.

In one embodiment of this and all other aspects described herein, the GATA1 binding element site is at chr2:60,495,264-60,495,271 in genome version hg38. In one embodiment, the GATA1 binding element comprises, consists of, or consists essentially of SEQ ID NO: 2. The GATA1 binding motif is further described in, e.g., Canver, M. C. et al. BCL11A enhancer dissection by Cas9-mediated in situ saturating mutagenesis. Nature 527, 192-197 (2015), which is incorporated herein by reference in its entirety.

SEQ ID NO: 2 is a nucleic acid sequence that encodes the GATA1 binding element. Within SEQ ID NO: 2, W=A or T and R=G or A

(SEQ ID NO: 2)
WGATAAR,

In one embodiment of this and all other aspects described herein, the deletion is of other functional sequences with the +58 BCL11A enhancer genomic region.

In another embodiment of this aspect and all other aspects described herein, the deletion comprises one or more of the DNAse 1-hypersensitive sites (DHS) +62, +58, and +55 (see e.g., US 2021-0115420, the contents of which are incorporated herein by reference in its entirety; Canver, M. C. et al. BCL11A enhancer dissection by Cas9-mediated in situ saturating mutagenesis. Nature 527, 192-197 (2015)). In another embodiment of this aspect and all other aspects described herein, the deletion consists essentially of one or more of the DNAse 1-hypersensitive sites (DHS) +62, +58, and +55. In another embodiment, the deletion consists of one or more of the DNAse 1-hypersensitive sites (DHS) +62, +58, and +55.

In another embodiment of this aspect and all other aspects described herein, the deletion is from 60,716,189 to 60,728,612, from 60,716,189 to 60,723,870, from 60,722,992 to 60,728,612, from 60,717,236 to 60,719,036, from 60,722,006 to 60,723,058, from 60,724,917 to 60,726,282, from 60,616,396 to 60,618,032, from 60,623,536 to 60,624,989, from 60,626,565 to 60,628,177, from 60,717,236 to 60,719,036, from 60,721,212 to 60,722,958, from 60,724,780 to 60,726,471, from 60,739,075 to 60,740,154, from 60,748,003 to 60,749,009, from 60,826,438 to 60,827,601, or from 60,831,589 to 60,833,556.

### Induced Pluripotent Stem Cells

In some embodiments, the genetic engineered human cells (e.g., genome modified hematopoietic stem or progenitor cells) described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein. Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others. Such reprogramming enhancers and others in the art are known to those of skill in the art and are not described in detail herein.

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, an hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Natl); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

### Genome Editing and DNA-Targeting Endonucleases

As used herein, the term “genome editing” refers to a reverse genetics method using artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point.

Genomic modification is typically performed using one or more of meganucleases, Zinc finger nucleases (ZFNs), Cas9/CRISPR system, and/or transcription-activator like effector nucleases (TALENs).

Meganucleases are commonly grouped into four families: the LAGLIDADG (SEQ ID NO: 4) family, the GIY-YIG family, the His-Cys box family and the HNH family. These families are characterized by structural motifs, which affect catalytic activity and recognition sequence. For instance, members of the LAGLIDADG (SEQ ID NO: 4) family are characterized by having either one or two copies of the conserved LAGLIDADG motif (SEQ ID NO: 4) (see Chevalier et al. (2001), Nucleic Acids Res. 29(18): 3757-3774). The LAGLIDADG (SEQ ID NO: 4) meganucleases with a single copy of the LAGLIDADG motif (SEQ ID NO: 4) form homodimers, whereas members with two copies of the LAGLIDADG (SEQ ID NO: 4) are found as monomers. Similarly, the GIY-YIG family members have a GIY-YIG module, which is 70-100 residues long and includes four or five conserved sequence motifs with four invariant residues, two of which are required for activity (see Van Roey et al. (2002), Nature Struct. Biol. 9: 806-811). The His-Cys box meganucleases are characterized by a highly conserved series of histidines and cysteines over a region encompassing several hundred amino acid residues (see Chevalier et al. (2001), Nucleic Acids Res. 29(18): 3757-3774). In the case of the NHN family, the members are defined by motifs containing two pairs of conserved histidines surrounded by asparagine residues (see Chevalier et al. (2001), Nucleic Acids Res. 29(18): 3757-3774). The four families of meganucleases are widely separated from one another with respect to conserved structural elements and, consequently, DNA recognition sequence specificity and catalytic activity.

Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision BioSciences' Directed Nuclease Editor™ genome editing technology.

ZFNs and TALENs restriction endonuclease technology utilizes a non-specific DNA cutting enzyme which is linked to a specific DNA sequence recognizing peptide(s) such as zinc fingers and transcription activator-like effectors (TALEs). Typically, an endonuclease whose DNA recognition site and cleaving site are separate from each other is selected and the its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, CA).

In some embodiments, the genomic modification is performed using a CRISPR/Cas system (see CRISPR/Cas Systems section below). Clustered regularly interspaced short palindromic repeats (CRISPR)/CRISPR-associated (Cas) systems is useful for RNA-programmable genome editing (see e.g., Jinek, M. et al. Science (2012) 337(6096):816-821).

Alternatively, genome editing can be performed using recombinant adeno-associated virus (rAAV) based genome engineering, which is a genome-editing platform centered around the use of rAAV vectors that enables insertion, deletion or substitution of DNA sequences into the genomes of live mammalian cells. The rAAV genome is a single-stranded deoxyribonucleic acid (ssDNA) molecule, either positive- or negative-sensed, which is about 4.7 kilobase long. These single-stranded DNA viral vectors have high transduction rates and have a unique property of stimulating endogenous homologous recombination in the absence of causing double strand DNA breaks in the genome. One of skill in the art can design a rAAV vector to target a desired genomic locus and perform both gross and/or subtle endogenous gene alterations in a cell, such as a deletion. rAAV genome editing has the advantage in that it targets a single allele and does not result in any off-target genomic alterations. rAAV genome editing technology is commercially available, for example, the rAAV GENESIS™ system from Horizon™ (Cambridge, UK).

### CRISPR/Cas Systems

In general, “a CRISPR/Cas system” refers collectively to transcripts and other elements involved in the expression of or direction of the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In some embodiments, the CRISPR/Cas system involves a ‘base editing system’ using modified conventional Cas endonucleases to change specific bases without cutting both strands of DNA.

A CRISPR system is typically characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination. In one embodiment, the “target” sequence can be the BCL11A gene, associated regulatory regions or GATA binding site.

Methods for CRISPR/Cas mediated genomic modification are known in the art and are not described in detail herein.

In one embodiment, the CRISPR enzyme is a Cas protein. Non-limiting examples of Cas proteins include Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c homologs thereof, or modified versions thereof. These enzymes are known; for example, the amino acid sequence of S. pyogenes Cas9 protein can be found in the SwissProt database under accession number Q99ZW2. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, the CRISPR enzyme can comprise at least one nuclear localization signal sequences (NLSs). In some embodiments, the CRISPR enzyme comprises at least one NLSs at or near the amino-terminus, at least one NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. Typically, an NLS consists of one or more short sequences of positively charged lysines or arginines exposed on the protein surface, but other types of NLS are known.

The sequence of the guide RNA (e.g., the sequence homologous to the target gene of interest) can be determined for the intended use. For example, to target the bcl11a gene, one would choose a guide RNA that targets and hybridize to bcl11a in a manner that effectively results in the desired alteration of the gene's expression. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything) has a single occurrence in the genome. A unique target sequence in a genome may include an S. pyogenes Cas9 target site of the form MMMMMMMMMNNNNNNNNNNNXGG where NNNNNNNNNNNXGG (N is A, G, T, or C; and X can be anything) has a single occurrence in the genome. Alternatively, the first 8 positions in the above mentioned unique sequences can be NNNNNNNN, for example, NNNNNNNNNNNNNNNNNNNNXGG.

In one embodiment, the guide RNA comprises BCL11a sgRNA 1617 of SEQ ID NO: 1 (e.g., CTAACAGTTGCTITfATCAC). This guide RNA is being tested in clinical trials and has proven efficacy for the induction of fetal hemoglobin. A subset of patients having the SNP polymorphism at site rs114518452 (i.e., cytosine (C) at that site) are at an increased risk of off-target effects, or genotoxicity (e.g., via chromosomal inversions or other deleterious mutations or rearrangement).

Where a subject is determined to have a SNP polymorphism at site rs114518452; (i.e., cytosine nucleotide at that site), the use of a high-fidelity Cas enzyme that lacks detectable off-target effects is necessary for safe and efficacious treatment of the subject. In one embodiment, the high-fidelity Cas enzyme does not substantially introduce DNA strand breaks at the site of the polymorphism. In other embodiments, the high-fidelity nuclease reduces the production of off-target indels by at least 10%, at least 20%, at least 30%, at least 40%, at least 50%, at least 60%, at least 70%, at least 80%, at least 90%, at least 100%, at least 2-fold, at least 5-fold, at least 10-fold, at least 50-fold, or at least 100-fold reduction as compared to the same gene editing system using a low-fidelity nuclease. An exemplary high-fidelity Cas enzyme without off-target effects includes SpCas9-HF1 (Kleinstiver et al. Nature 2016; 529(7587):490-495, the contents of which are incorporated herein by reference in its entirety) or Cas9HiFi. Such high-fidelity Cas enzymes or expression vectors comprising the same are available commercially from e.g., ThermoFisher Scientific (Waltham, MA), Novatein Biosciences (Woburn, MA), and AddGene (Watertown, MA), among others.

### Pharmaceutically Acceptable Carriers

The methods of administering human hematopoietic progenitors to a subject as described herein involve the use of therapeutic compositions comprising hematopoietic progenitor cells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In general, the hematopoietic progenitor cells described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the hematopoietic progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

### Administration & Efficacy

As used herein, the terms “administering,” “introducing” and “transplanting” are used interchangeably in the context of the placement of cells, e.g. hematopoietic progenitor cells, as described herein into a subject, by a method or route which results in at least partial localization of the introduced cells at a desired site, such as the bone marrow or blood, such that a desired effect(s) is produced. The cells e.g. hematopoietic progenitor cells, or their differentiated progeny can be administered by any appropriate route which results in delivery to a desired location in the subject where at least a portion of the implanted cells or components of the cells remain viable and/or engraft in the subject. The period of viability of the cells after administration to a subject can be as short as a few hours, e.g., twenty-four hours, to a few days, to as long as several years, i.e., long-term engraftment. For example, in some embodiments of the aspects described herein, an effective amount of hematopoietic progenitor cells is administered via a systemic route of administration, such as an intraperitoneal or intravenous route.

When provided prophylactically, hematopoietic progenitor cells described herein can be administered to a subject in advance of any symptom of a hemoglobinopathy, e.g., prior to the switch from fetal γ-globin to predominantly β-globin (e.g., in a fetus, neonate, infant, toddler or child). Accordingly, the prophylactic administration of a hematopoietic progenitor cell population serves to prevent a hemoglobinopathy, as disclosed herein.

When provided therapeutically, hematopoietic progenitor cells are provided at (or after) the onset of a symptom or indication of a hemoglobinopathy, e.g., upon the onset of sickle cell disease.

In some embodiments of the aspects described herein, the hematopoietic progenitor cell population being administered according to the methods described herein comprises allogeneic hematopoietic progenitor cells obtained from one or more donors. As used herein, “allogeneic” refers to a hematopoietic progenitor cell or biological samples comprising hematopoietic progenitor cells obtained from one or more different donors of the same species, where the genes at one or more loci are not identical. For example, a hematopoietic progenitor cell population being administered to a subject can be derived from umbilical cord blood obtained from one more unrelated donor subjects, or from one or more non-identical siblings. In some embodiments, syngeneic hematopoietic progenitor cell populations can be used, such as those obtained from genetically identical animals, or from identical twins. In other embodiments of this aspect, the hematopoietic progenitor cells are autologous cells; that is, the hematopoietic progenitor cells are obtained or isolated from a subject and administered to the same subject, i.e., the donor and recipient are the same.

For use in the various aspects described herein, an effective amount of hematopoietic progenitor cells, comprises at least 10² hematopoietic progenitor cells, at least 5×10² hematopoietic progenitor cells, at least 10³ hematopoietic progenitor cells, at least 5×10³ hematopoietic progenitor cells, at least 10⁴ hematopoietic progenitor cells, at least 5×10⁴ hematopoietic progenitor cells, at least 10⁵ hematopoietic progenitor cells, at least 2×10⁵ hematopoietic progenitor cells, at least 3×10⁵ hematopoietic progenitor cells, at least 4×10⁵ hematopoietic progenitor cells, at least 5×10⁵ hematopoietic progenitor cells, at least 6×10⁵ hematopoietic progenitor cells, at least 7×10⁵ hematopoietic progenitor cells, at least 8×10⁵ hematopoietic progenitor cells, at least 9×10⁵ hematopoietic progenitor cells, at least 1×10⁶ hematopoietic progenitor cells, at least 2×10⁶ hematopoietic progenitor cells, at least 3×10⁶ hematopoietic progenitor cells, at least 4×10⁶ hematopoietic progenitor cells, at least 5×10⁶ hematopoietic progenitor cells, at least 6×10⁶ hematopoietic progenitor cells, at least 7×10⁶ hematopoietic progenitor cells, at least 8×10⁶ hematopoietic progenitor cells, at least 9×10⁶ hematopoietic progenitor cells, or multiples thereof. The hematopoietic progenitor cells can be derived from one or more donors, or can be obtained from an autologous source. In some embodiments of the aspects described herein, the hematopoietic progenitor cells are expanded in culture prior to administration to a subject in need thereof. It will be understood by those of skill in the art that in some embodiments, therapeutic hematopoietic stem or progenitor cells are pre-treated to inhibit BCL11A and/or express fetal hemoglobin isoforms.

In one embodiment, the term “effective amount” as used herein refers to the amount of a population of human hematopoietic progenitor cells or their progeny needed to alleviate at least one or more symptom of a hemoglobinopathy, and relates to a sufficient amount of a composition to provide the desired effect, e.g., treat a subject having a hemoglobinopathy. The term “therapeutically effective amount” therefore refers to an amount of hematopoietic progenitor cells or a composition comprising hematopoietic progenitor cells that is sufficient to promote a particular effect when administered to a typical subject, such as one who has or is at risk for a hemoglobinopathy. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

As used herein, “administered” refers to the delivery of a hematopoietic stem cell composition as described herein into a subject by a method or route which results in at least partial localization of the cell composition at a desired site. A cell composition can be administered by any appropriate route which results in effective treatment in the subject, i.e. administration results in delivery to a desired location in the subject where at least a portion of the composition delivered, i.e. at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. For the delivery of cells, administration by injection or infusion is generally preferred.

In one embodiment, the cells as described herein are administered systemically. The phrases “systemic administration,” “administered systemically”, “peripheral administration” and “administered peripherally” as used herein refer to the administration of a population of hematopoietic progenitor cells other than directly into a target site, tissue, or organ, such that it enters, instead, the subject's circulatory system and, thus, is subject to metabolism and other like processes.

The efficacy of a treatment comprising a composition as described herein for the treatment of a hemoglobinopathy can be determined by the skilled clinician. However, a treatment is considered “effective treatment,” as the term is used herein, if any one or all of the signs or symptoms of, as but one example, levels of fetal β-globin are altered in a beneficial manner, other clinically accepted symptoms or markers of disease are improved or ameliorated, e.g., by at least 10% following treatment with an inhibitor. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of sepsis; or (2) relieving the disease, e.g., causing regression of symptoms; and (3) preventing or reducing the likelihood of the development of infection or sepsis.

The treatment according to the methods provided herein can reduce or eliminate one or more symptoms associated with a β-globin disorder by increasing the amount of fetal hemoglobin in the individual. Symptoms typically associated with a hemoglobinopathy, include for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

In one embodiment, any method known in the art can be used to measure an increase in fetal hemoglobin expression, e.g., Western Blot analysis of fetal hemoglobin protein and quantifying mRNA of fetal γ-globin.

In some embodiments, a therapeutic composition as described herein comprises a hematopoietic stem cell that comprises a genomic modification to inhibit BCL11A. Following in vitro or ex vivo cell culture, isolation, or differentiation as described herein, engineered cells are prepared for treatment and/or implantation. The cells are suspended in a physiologically compatible carrier, such as cell culture medium (e.g., Eagle's minimal essential media), phosphate buffered saline, or a hematopoietic stem cell specific medium. The volume of cell suspension to be implanted will vary depending on the site of implantation, treatment goal, and cell density in the solution.

In some embodiments, the engineered cell composition comprises at least 1,000, at least 10,000, at least 100,000, at least 10⁶, at least 10⁷, at least 10³, at least 10⁹, at least 10¹⁰ engineered cells or more are administered to a subject for the treatment of a hemoglobinopathy. In one embodiment, the engineered cell composition comprises at least 100,000 engineered cells as described herein. It will be appreciated by one of skill in the art that the number of cells can be tailored or optimized for efficacious treatment of the subject in need thereof. As such, one of skill in the art can perform dose escalation studies, if desired.

It will be appreciated by one of skill in the art that a cell composition useful for treating a hemoglobinopathy does not need to be a pure, homogeneous culture of e.g., genetically altered hematopoietic stem or progenitor cells. Accordingly, in one embodiment, the composition administered comprises at least 2% engineered cells (e.g., engineered hematopoietic stem cells that produce fetal hemoglobin). In other embodiments, the composition comprises at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more engineered cells as described herein.

The cells can be administered to a subject by any appropriate route that results in delivery of the cells to a desired location in the subject where at least a portion of the cells remain viable. It is preferred that at least 5% remain viable. In other embodiments, at least 10%, at least 20%, at least 30%, at least 40%, or at least 50%, at least 60%, at least 70%, at least 80%, at least 90%, at least 95%, at least 98%, at least 99% or more of the cells remain viable after administration into a subject. The period of viability of the cells after administration to a subject can be as short as a few hours, e.g., twenty-four hours, to a few days, to as long as a few weeks to months.

To accomplish these methods of administration, the engineered cell composition(s) can be inserted into a delivery device that facilitates introduction by injection or implantation of the cells into the subject. Typically, the cells are injected into the target area as a cell suspension. Alternatively, the engineered cells can be embedded in a solid or semisolid support matrix when contained in such a delivery device.

In some embodiments, administration of a composition comprising engineered cells is repeated after a given interval of time (e.g., one day, three days, one week, two weeks, three weeks, one month or more. Repeated treatments can be performed, for example, to establish or maintain a threshold level of engraftment necessary to continue effective treatment, as necessary, of a hemoglobinopathy. In some embodiments, the method is repeated twice, three times, four times, five times or more.

### Kits

In one aspect, provided herein are kits containing any one or more of the elements disclosed in the above methods and compositions. In one embodiment, the kit comprises, consists of, or consists essentially reagents and instructions for assessing the presence or absence of a SNP polymorphism. In another embodiment, the kit further comprises a guide RNA. In some embodiments, the guide RNA is BCL11A sgRNA 1617. In another embodiment, the kit further comprises a component of the CRISPR system. In some embodiments, the kit can comprise a high-fidelity Cas enzyme for use with BCL11A sgRNA 1617 in those patients having the polymorphism at site rs114518452 (i.e., cytosine on the 5′ to 3′ strand).

Another aspect provided herein relates to a kit comprising a means for determining the presence or absence of a variant allele at rs114518452 wherein the presence of said variant allele (i.e., C at that site on the 5′ to 3′ strand) at said polymorphic site indicates that the subject should be treated with any desired method excluding treatment with CRISPR/Cas comprising a guide RNA of SEQ ID NO: 1.

The kit can comprise the components necessary to determine the presence or absence of said variant allele including PCR primers (such as allele specific primers) and/or probes, PCR enzymes, restriction enzymes, and DNA or RNA purification means. The kit may contain at least one pair of primers, or probes.

Other components include labeling means, buffers for the reactions. In addition, a control nucleic acid sample may be included, which comprises a wild type or variant nucleic acid sequence as defined above, or a PCR product of the same. The kit will usually also comprise instructions for carrying out the methods as described herein and a key detailing the correlation between the results and the use of a guide RNA of SEQ ID NO: 1 in a CRISPR/Cas genomic modification system.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

This invention is further illustrated by the following example which should not be construed as limiting. The contents of all references cited throughout this application, as well as the figures and table are incorporated herein by reference.

The invention may be as described in any one of the following numbered paragraphs:
1. A method for enhancing levels of a fetal hemoglobin isoform in a subject in need thereof, the method comprising: (i) receiving results of a SNP polymorphism assay from a biological sample obtained from a subject indicating the absence of a SNP polymorphism at site rs114518452, (ii) modifying the genome of at least one hematopoietic stem cell to inhibit BCL11A, and (iii) administering the modified hematopoietic stem cell of step (ii) to the subject in need thereof, thereby enhancing levels of a fetal hemoglobin isoform in the subject.
2. A method for treating a hemoglobinopathy in a subject, the method comprising: (i) receiving results of a SNP polymorphism assay from a biological sample obtained from a subject indicating the absence of a SNP polymorphism at site rs114518452, (ii) modifying the genome of at least one hematopoietic stem cell to inhibit BCL11A, and (iii) administering the modified hematopoietic stem cell of step (ii) to a subject having a hemoglobinopathy, thereby treating the hemoglobinopathy in the subject.
3. The method of paragraph 1 or 2, wherein the fetal hemoglobin isoform comprises a γ subunit.
4. The method of paragraph 1 or 3, wherein the subject in need thereof comprises a hemoglobinopathy.
5. The method of any one of paragraphs 1-4, wherein the hemoglobinopathy comprises a sickle cell disease.
6. The method of paragraph 5, wherein the sickle cell disease is sickle cell anemia, sickle-hemoglobin C disease (HbSC), sickle beta-plus-thalassaemia (HbS/β+), or sickle beta-zero-thalassaemia (HbS/β0).
7. The method of any one of paragraphs 2-6, wherein the hemoglobinopathy is a β-thalassemia.
8. The method of any one of paragraphs 1-7, further comprising a step of analyzing the subject's genome for a SNP polymorphism at site rs114518452.
9. The method of any one of paragraphs 1-8, wherein the step of modifying the genome comprises contacting the hematopoietic stem cell with a CRISPR/Cas genome modification system and a BCL11A specific guide RNA.
10. The method of paragraph 9, wherein the BCL11A specific guide RNA comprises BCL11A sgRNA 1617 having a sequence of: CTAACAGTTGCTITTATCAC (SEQ ID NO: 1).
11. The method of any one of paragraphs 1-10, wherein the hematopoietic stem cell is isolated from the subject and step (ii) is performed ex vivo.
12. The method of any one of paragraphs 1-11, wherein hematopoietic stem cell is isolated from a donor and step (ii) is performed ex vivo.
13. The method of any one of paragraphs 1-12, wherein hematopoietic stem cell is derived from an induced pluripotent stem cell or an embryonic stem cell.
14. The method of any one of paragraphs 1-13, wherein the nucleotide base at site rs114518452 is guanine (G) on the 5′ to 3′ strand.
15. A method for selecting a guide RNA for a CRISPR/Cas based hemoglobinopathy treatment for a subject in need thereof, the method comprising: (i) receiving results of a SNP polymorphism assay from a biological sample obtained from a subject indicating the absence of a SNP polymorphism at site rs114518452, (ii) selecting BCL11A sgRNA 1617 as a guide RNA for use with a CRISPR/Cas based hemoglobinopathy treatment.
16. The method of paragraph 15, wherein the nucleotide base at site rs114518452 is guanine (G) on the 5′ to 3′ strand.
17. The method of paragraph 15 or 16, further comprising a step of analyzing the subject's genome for a SNP polymorphism at site rs114518452.
18. A method for selecting a guide RNA for a CRISPR/Cas based hemoglobinopathy treatment for a subject in need thereof, the method comprising: (i) receiving results of a SNP polymorphism assay from a biological sample obtained from a subject indicating the presence of a SNP polymorphism at site rs114518452, (ii) excluding BCL11A sgRNA 1617 as a guide RNA for use with a CRISPR/Cas based hemoglobinopathy treatment.
19. The method of paragraph 18, wherein the nucleotide base at site rs114518452 is cytosine (C) on the 5′ to 3′ strand.
20. The method of paragraph 18 or 19, further comprising a step of analyzing the subject's genome for a SNP polymorphism at site rs114518452.
21. A method for optimizing a CRISPR/Cas based hemoglobinopathy treatment for a subject in need thereof, the method comprising: receiving results of a SNP polymorphism assay from a biological sample obtained from a subject indicating the presence of a SNP polymorphism at site rs114518452 and (a) excluding BCL11A sgRNA 1617 as a guide RNA for use with a CRISPR/Cas based hemoglobinopathy treatment, (b) selecting BCL11A sgRNA 1617 as a guide RNA for use with a high-fidelity Cas enzyme.
22. The method of paragraph 21, wherein the high-fidelity Cas enzyme comprises Cas9 R691A.
23. The method of paragraph 21 or 22, wherein the SNP polymorphism at site rs114518452 comprises cytosine on the 5′ to 3′ strand.
24. A hematopoietic stem cell composition comprising a hematopoietic stem cell modified to inhibit BCL11A for use in a method of treating a subject with a hemoglobinopathy, wherein the method comprises receiving results of a SNP polymorphism assay from a biological sample obtained from the subject indicating the absence of a SNP polymorphism at site rs114518452.

### EXAMPLES

The following provides non-limiting Examples demonstrating and supporting the technology as described herein.

The methods and compositions described herein detail fetal hemoglobin reinduction using Cas9 fusion proteins. The invention methods and compositions described herein represent an extension of previously described Cas9-fusion proteins with applications for human hematopoietic stem and progenitor cells (CD34+ HSPCs). The methods and compositions provided herein are a therapeutic treatment for beta-hemoglobinopathies, such as sickle cell disease and beta-thalassemia. The methods and compositions described herein detail the reactivating gamma-globin expression to complement the loss of beta-globin function.

### Example 1: CRISPRme Identification of Off-Target Effects

CRISPR genome editing offers unprecedented opportunities to develop novel therapeutics by introducing targeted genetic or epigenetic modifications to genomic regions of interest. Briefly, CRISPR offers an easy and programmable platform that couples binding to a genomic target sequence of choice with diverse effector proteins through RNA:DNA (spacer:protospacer) complementary sequence interactions mediated by a guide RNA (gRNA) restricted by protospacer adjacent motif (PAM) sequences. Editing effectors may consist of nucleases to introduce targeted double strand breaks leading to short indels and templated repairs (e.g. Cas9), deaminases for precise substitutions (base editors), or chromatin regulators for transcriptional interference or activation (CRISPRi/a) among others to achieve a range of desired biological outcomes¹.

CRISPR based systems may create unintended off-target modifications posing potential genotoxicity for therapeutic use. Several experimental assays and computational methods are available to uncover or forecast these off-targets². Off-target sites are partially predictable based on homology to the target site protospacer and PAM sequence. Beyond the number of mismatches or bulges, a variety of sequence features, like position of mismatch or bulge with respect to PAM or specific base changes, contribute to off-target potential²⁻⁵. Computational models can complement experimental approaches to off-target nomination in several respects: to triage gRNAs prior to experiments by predicting the number and cleavage potential of off-target sites; to prioritize target sites for experimental scrutiny; and to supplement experimental approaches in nominating sites for sequence validation. Genetic variants may alter protospacer and PAM sequences and therefore may influence both on-target and off-target potential. Although a variety of in vitro and cell-based experimental methods can be used to empirically nominate off-target sites, these methods either use homology to the reference genome as a criteria to define the search space and/or use a limited set of human donor genomes to evaluate off-target potential^{3,6}. Therefore, computational methods may be especially useful to predict the impact of off-target sequences not found in reference genomes.

Prior studies considering gRNAs targeting therapeutically relevant genes and population-based variant databases like the 1000 Genomes Project (1000G) and the Exome Aggregation Consortium have highlighted how genetic variants can significantly alter the off-target landscape by creating novel and personal off-target sites not present in a single reference genome^{7,8}. Although these prior studies provide code to reproduce analyses, implementation choices make these tools not suitable to analyze large variant datasets and to consider higher numbers of mismatches. In addition, these methods do not account for indel variants, ignore bulges between RNA:DNA hybrids, cannot efficiently model alternative haplotypes and require extensive computational skills to utilize.

Several user-friendly websites have been developed to aid the design of gRNAs and to assess their potential off-targets⁹⁻¹². Even though variant-aware prediction is an important problem for genome editing interventions, these scalable GUI-based tools do not account for genetic variants. In addition, these tools artificially limit the number of mismatches for the search and/or do not support DNA or RNA bulges. Therefore, designing gRNAs for therapeutic intervention using current widely available tools could miss important off-target sites that may lead to unwanted genotoxicity. A complete and exhaustive off-target search with an arbitrary number of mismatches, bulges, and genetic variants that is haplotype-aware is a computationally challenging problem that requires specialized and efficient data structures.

A command line tool that partially solves these challenges was previously generated called CRISPRitz¹³. This tool uses optimized data structures to efficiently account for single variants, mismatches and bulges but with significant limitations¹³. This work was substantially extended by developing CRISPRme, a tool to aid with the design of gRNAs with added support for haplotype-aware off-target enumeration, short indel variants and a flexible number of mismatches and bulges. CRISPRme is a unified, user-friendly web-based application that provides several reports to prioritize putative off-targets based on their risk in a population or individuals.

CRISPRme is flexible to accept user-defined genomic annotations, which could include empirically identified off-target sites or cell type specific chromatin features. It can integrate population genetic variants from sets of phased individual variants (like those from 1000 Genomes Project¹⁴), unphased individual variants (like those from the Human Genome Diversity Project, HGDP¹⁵) and population-level variants (like those from the Genome Aggregation Database, gnomAD¹⁶). Furthermore, it can accept personal genomes from individual subjects and identify and prioritize private off-targets due to variants specific to a single individual.

The utility of CRISPRme was tested by analyzing the off-target potential of a gRNA currently being tested in clinical trials for SCD and β-thalassemia¹⁷⁻¹⁹. The inventors identified possible off-targets introduced by genetic variants included within and extending beyond the 1000 Genomes Project. It is predicted that the most likely off-target site is introduced by an African ancestry prevalent (MAF African 4.5%) variant and provide experimental evidence of its off-target potential in gene edited human CD34+ hematopoietic stem and progenitor cells.

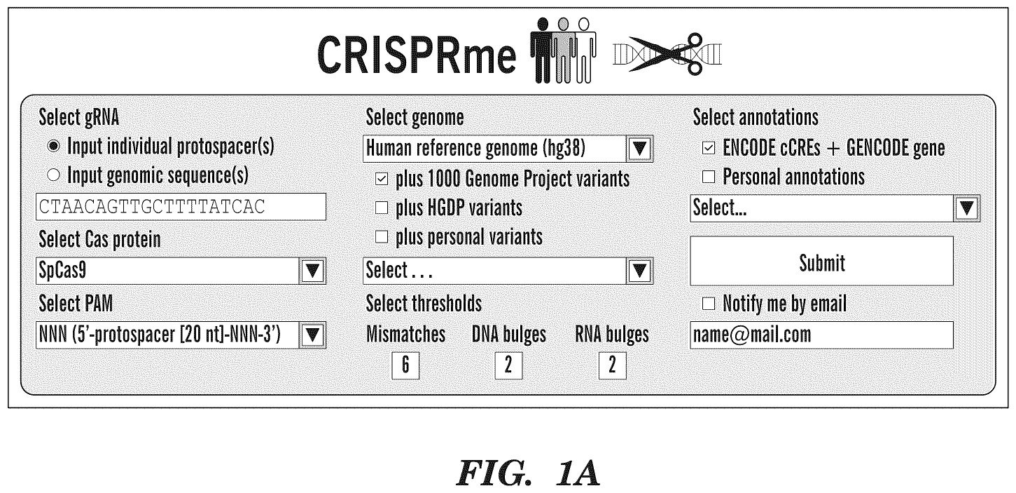

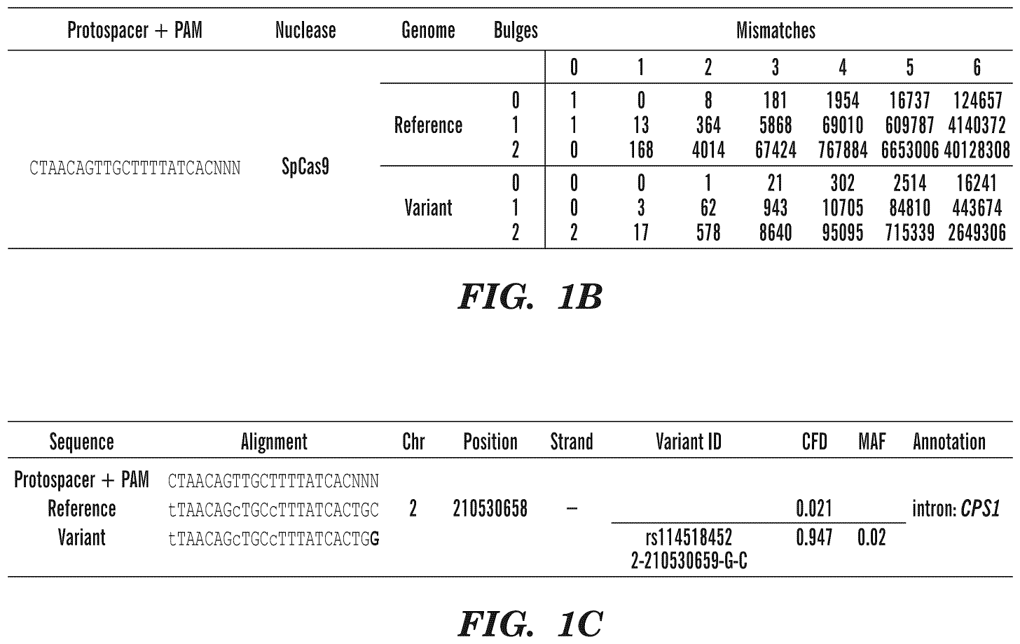

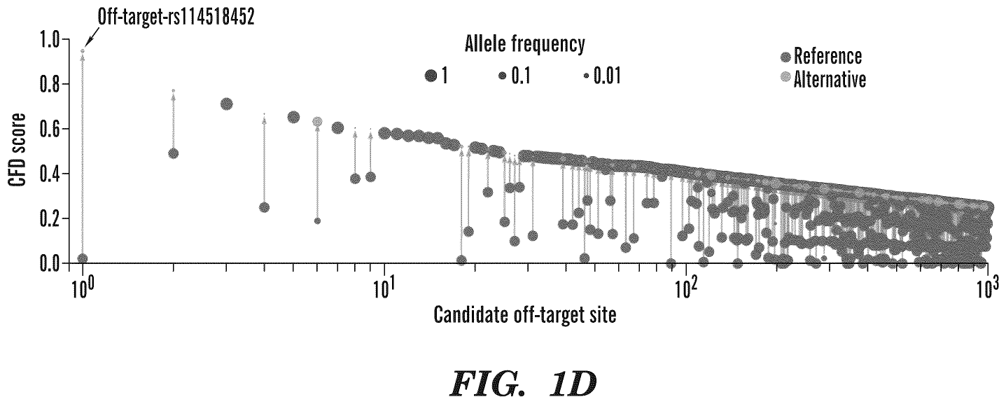

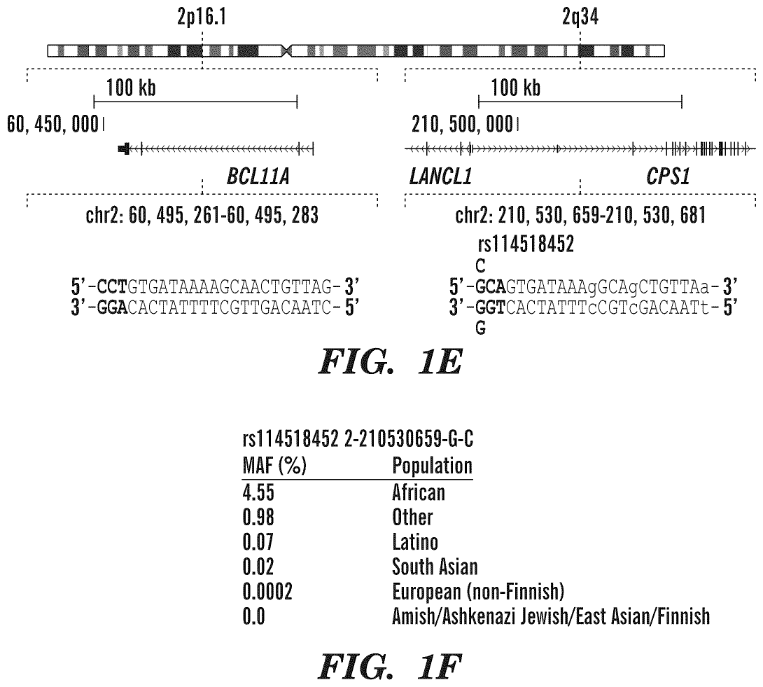

CRISPRme was used to assess off-target effects of a BCL11a gRNA (#1617) targeting a GATA1 binding motif at the +58 erythroid enhancer of BCL11A^{17,18}. A recent clinical report described two patients, one with SCD and one with β-thalassemia, each treated with autologous gene modified hematopoietic stem and progenitor cells (HSPCs) edited with Cas9 and this gRNA, who showed sustained increases in fetal hemoglobin, transfusion-independence and absence of vaso-occlusive episodes (in the SCD patient) following therapy¹⁹. This study as well as prior pre-clinical studies with the same gRNA (#1617) did not reveal evidence of off-target editing in treated cells considering off-target sites nominated by bioinformatic analysis of the human reference genome and empiric analysis of in vitro genomic cleavage potential^{18,19,22}. CRISPRme analysis found that the predicted off-target site with both the greatest CFD score and the greatest increase in CFD score between alternative and reference alleles was at an intronic sequence of CPS1 (FIGS. 1C and 1D), a genomic target subject to common genetic variation (modified by variant with 1000 Genomes Phase 3 MAF≥1%). CFD scores range from 0 to 1, where the on-target site has a score of 1. The alternative allele rs114518452-C generates a TGG PAM sequence (that is, the optimal PAM for SpCas9) for a potential off-target site with 3 mismatches with a CFD score (CFDalt 0.95) approaching that of the on-target site. In contrast, the reference allele rs114518452-G disrupts the PAM to TGC, which markedly reduces predicted cleavage potential (CFDref 0.02). rs114518452-C is present at 1.33% total allele frequency in gnomAD v3.116, with 4.55% frequency in African/African-American, 0.015% in European (non-Finnish) and 0.0% in East Asian super-populations (FIGS. 1E, 1F, and Table 1).

TABLE 1
Complete population frequencies
for rs114518452 from gnomAD v3.1¹⁶

| | Allele | Allele | Number of | Allele |
|---|---|---|---|---|
| Population | Count | Number | Homozygotes | Frequency |
| African/African- | 1882 | 41386 | 39 | 0.04547 |
| American | | | | |
| Other | 19 | 2090 | 0 | 0.009091 |
| Latino/Admixed | 100 | 15246 | 0 | 0.006559 |
| American | | | | |
| South Asian | 6 | 4830 | 0 | 0.001242 |
| European (non- | 10 | 67992 | 0 | 0.0001471 |
| Finnish) | | | | |
| European | 0 | 10612 | 0 | 0 |
| (Finnish) | | | | |
| Amish | 0 | 912 | 0 | 0 |
| East Asian | 0 | 5170 | 0 | 0 |
| Middle Eastern | 0 | 316 | 0 | 0 |
| Ashkenazi | 0 | 3470 | 0 | 0 |
| Jewish | | | | |
| XX | 1088 | 77776 | 23 | 0.01399 |
| XY | 929 | 74248 | 16 | 0.01251 |
| Total | 2017 | 152024 | 39 | 0.01327 |

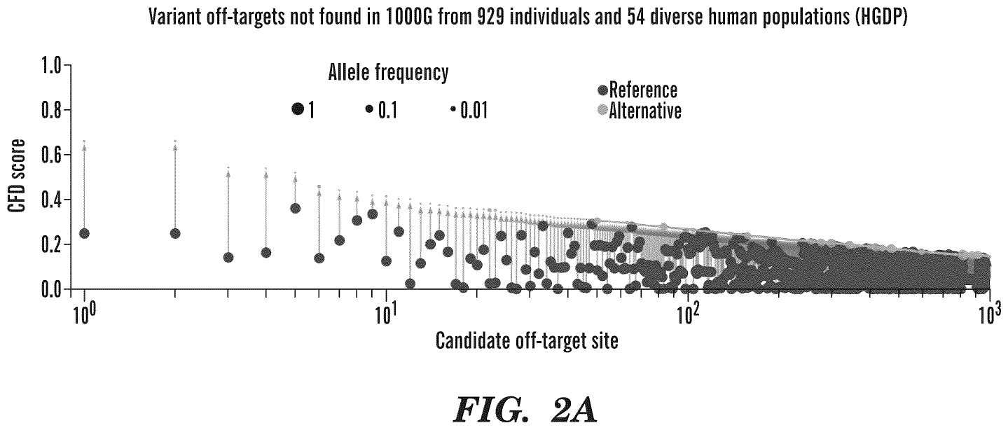

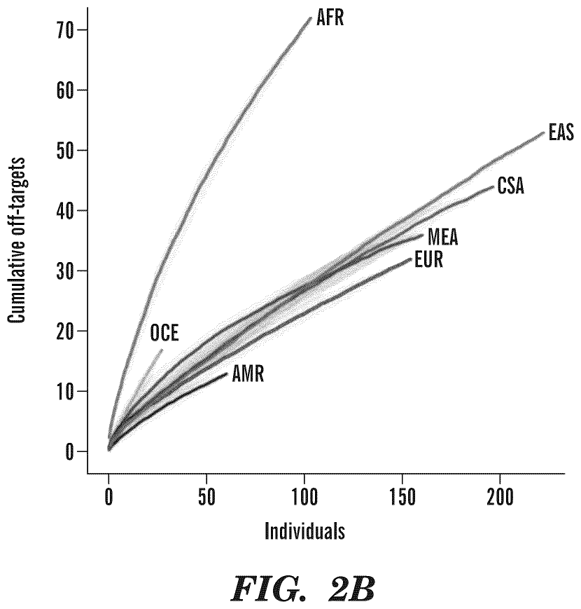

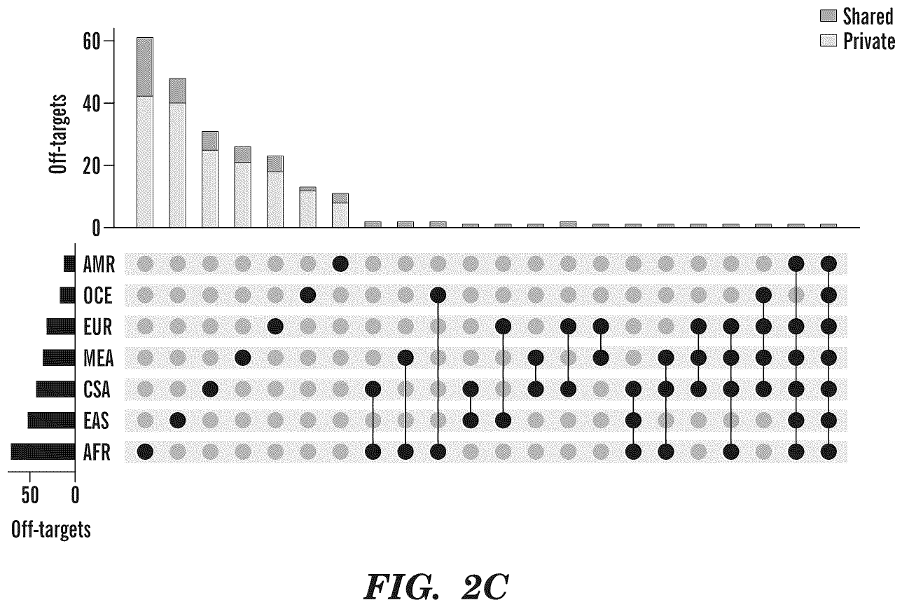

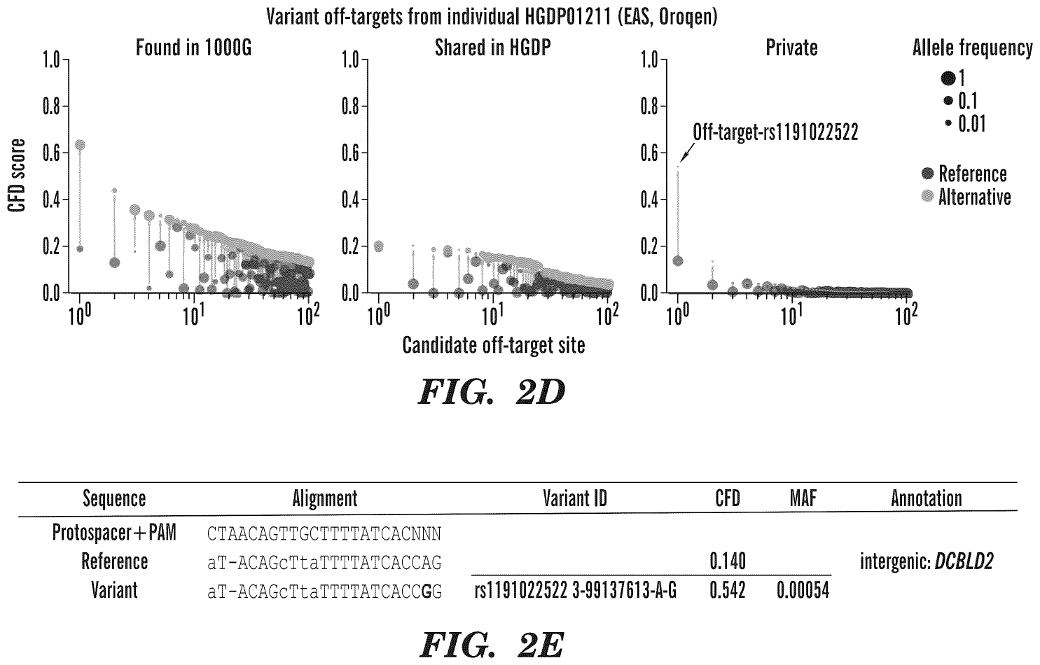

To consider the off-target potential that could be introduced by personal genetic variation that would not be predicted by 1000G variants, Human Genome Diversity Project (HGDP) variants identified from whole genome sequences of 929 individuals from 54 diverse human populations were analyzed¹⁵. There were 222 observed off-targets with CFD ≥0.2 for which the CFD score in HGDP exceeded that found in either the reference genome or due to a variant in 1000 Genomes by at least 0.1 (FIG. 2A). These additional variant off-targets not found from 1000 Genomes were observed in each super-population, with the greatest frequency in the African super-population (FIG. 2B). 204 (91.9%) of these variant off-targets not found by 1000 Genomes were unique to a super-population and 162 (73.0%) of these were unique to just one individual (FIG. 2C). Single individual focused searches, for example an analysis of HGDP01211, an individual of the Oroqen population within the East Asian super-population, showed that most variant off-targets (with higher CFD score than reference) were due to variants from 1000G (n=150754, 91.4%), a subset were due to variants shared with other individuals from HGDP but absent from 1000G (n=14184, 8.5%), and a small fraction were private to the individual (n=1120, 0.6%) (FIG. 2D). Among these private off-targets was one generated by a variant (rs1191022522, 3-99137613-A-G, MAF 0.00054) where the alternative allele produces an NGG PAM that increases the CFD score from 0.140 to 0.542 (FIGS. 2D and 2E).

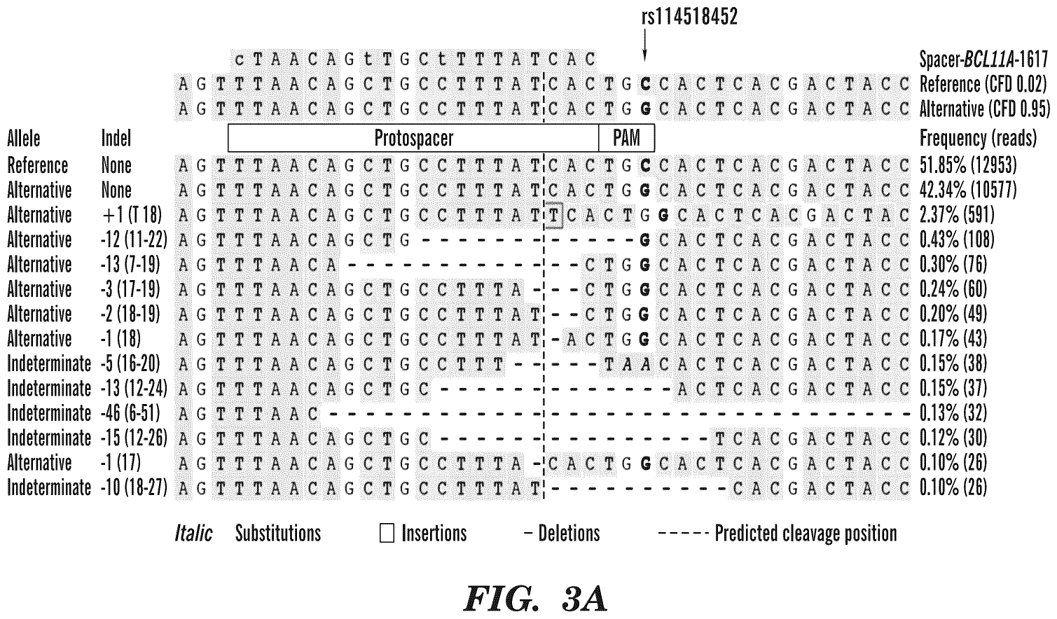

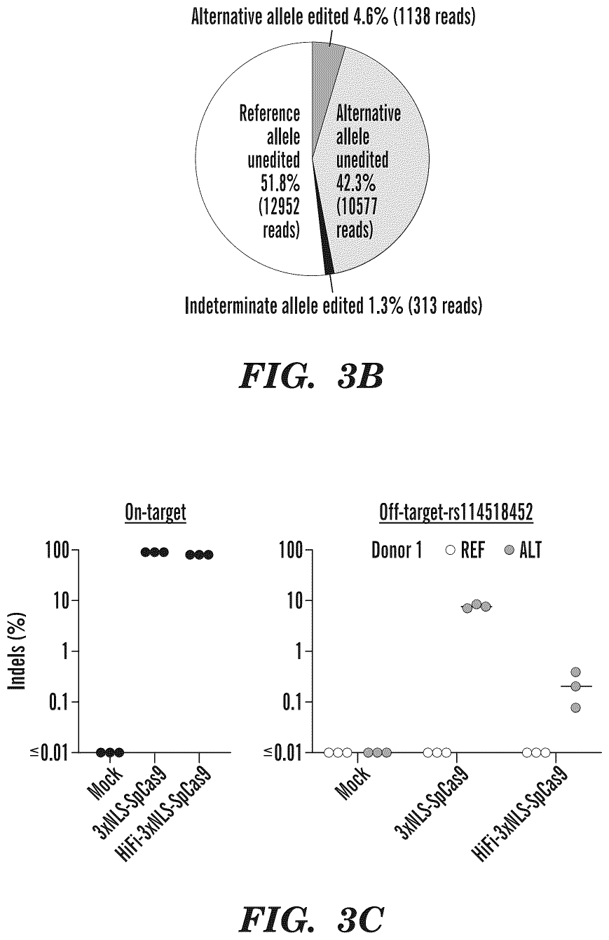

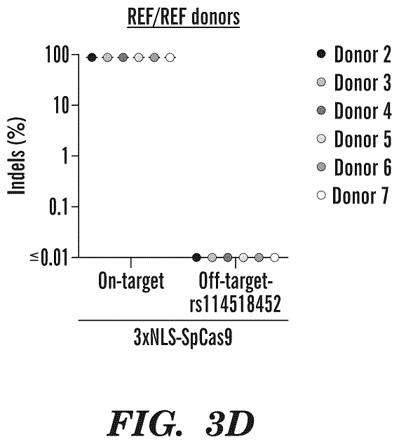

To validate the top predicted off-target by CRISPRme, a CD34+ HSPC donor of African ancestry heterozygous for rs114518452-G/C (the variant with the largest observed effect on CFD; FIG. 1E, 1F) was identified. RNP electroporation was performed using a gene editing protocol that preserves engrafting HSC function¹⁸. Amplicon sequencing analysis showed 92.0% indels at the on-target site and 4.8% indels at the off-target site. Evaluable indels were strictly found at the alternative PAM-creation allele without indels observed at the reference allele (FIGS. 3A-3C), indicating ˜9.6% off-target editing of the alternative allele. In an additional 6 HSPC donors homozygous for the reference allele rs114518452-G/G, no indels were observed, indicating strict restriction of off-target editing to the alternative allele (FIG. 3D).

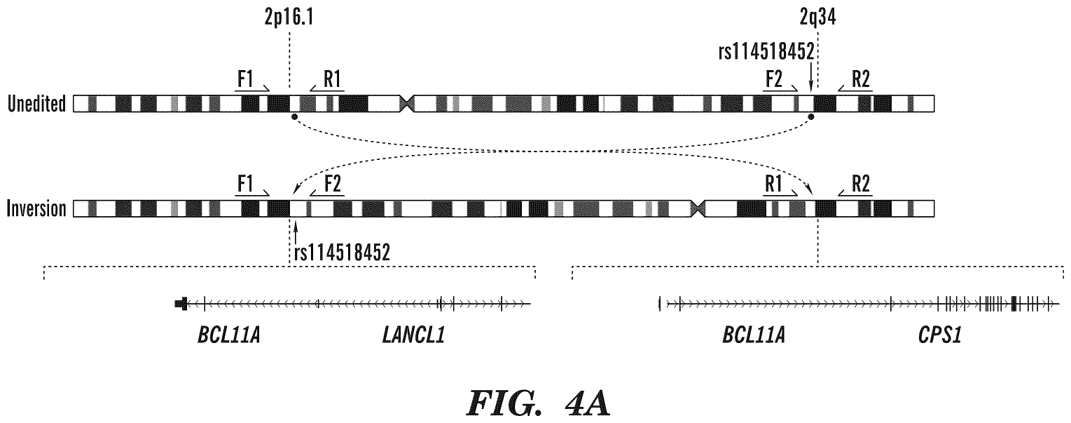

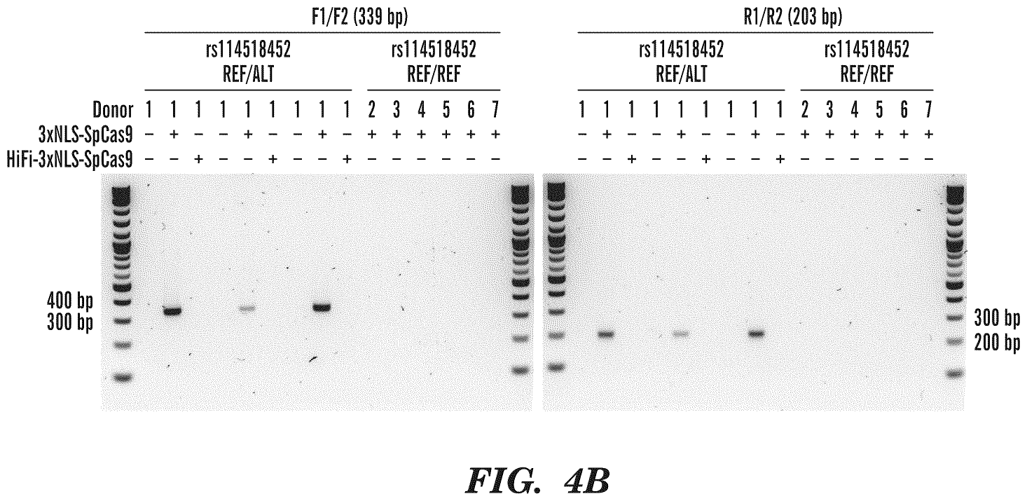

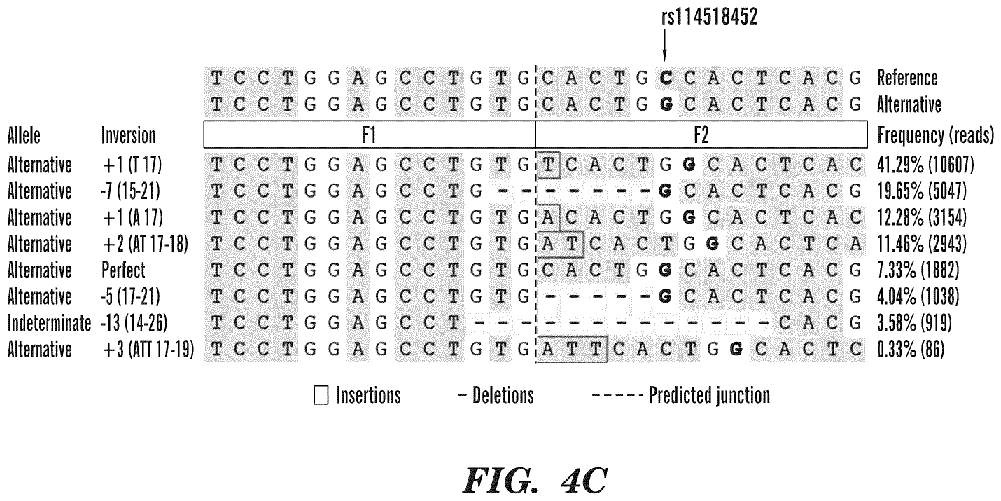

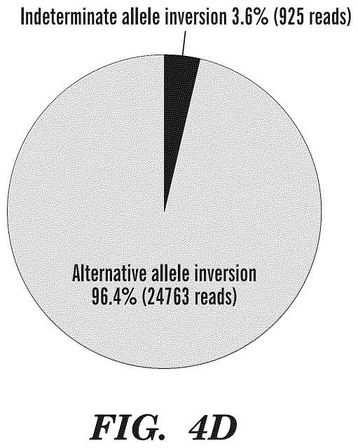

The on-target BCL11A intronic enhancer site is on chr2p and the off-target-rs114518452 site is on chr2q within an intron of a non-canonical transcript of CPS1. Inversion PCR demonstrated inversion junctions consistent with the presence of ˜150 Mb pericentric inversions between BCL11A and the off-target site only in edited HSPCs carrying the alternative allele (FIGS. 4A and 4B). Deep sequencing of inversion junctions showed that inversions were restricted to the alternative allele in the heterozygous cells (FIG. 4C, 4D). Gene editing following the same electroporation protocol using a HiFi variant 3×NLS-SpCas9 (R691A)²³ in heterozygous cells revealed 82.3% on-target indels with only 0.1% indels at the rs114518452-C off-target site, i.e. a ˜48-fold reduction compared to SpCas9 (FIG. 3c). Inversions were not detected following HiFi-3×NLS-SpCas9 editing (FIG. 4B).

These results demonstrate how personal genetic variation can influence the off-target potential of therapeutic gene editing. In the case of BCL11A enhancer editing, up to ˜10% of SCD patients with African ancestry are expected to carry at least one rs114518452-C allele. In general, therapeutic gene editing clinical trials can consider evaluating the impact of population and private genetic variation on gene editing outcomes including individual patient assessment and monitoring. CRISPRme offers a simple-to-use tool to comprehensively evaluate off-target potential across diverse populations and within individuals. CRISPRme, available at http://crisprme.di.univr.it, can be deployed locally to preserve privacy.

### REFERENCES

1. Anzalone, A. V., Koblan, L. W. & Liu, D. R. Genome editing with CRISPR-Cas nucleases, base editors, transposases and prime editors. Nat. Biotechnol. 38, 824-844 (2020).
2. Clement, K., Hsu, J. Y., Canver, M. C., Joung, J. K. & Pinello, L. Technologies and Computational Analysis Strategies for CRISPR Applications. Mol. Cell 79, 11-29 (2020).
3. Bao, X. R., Pan, Y., Lee, C. M., Davis, T. H. & Bao, G. Tools for experimental and computational analyses of off-target editing by programmable nucleases. Nat. Protoc. 16, 10-26(2021).
4. Hsu, P. D. et al. DNA targeting specificity of RNA-guided Cas9 nucleases. Nat. Biotechnol. 31, 827-832 (2013).
5. Doench, J. G. et al. Optimized sgRNA design to maximize activity and minimize off-target effects of CRISPR-Cas9. Nat. Biotechnol. 34, 1-12 (2016).
6. Chaudhari, H. G. et al. Evaluation of Homology-Independent CRISPR-Cas9 Off-Target Assessment Methods. CRISPR J 3, 440-453 (2020).
7. Lessard, S. et al. Human genetic variation alters CRISPR-Cas9 on- and off-targeting specificity at therapeutically implicated loci. Proc. Natl. Acad. Sci. U.S.A. (2017) doi:10.1073/pnas.1714640114.
8. Scott, D. A. & Zhang, F. Implications of human genetic variation in CRISPR-based therapeutic genome editing. Nat. Med. (2017) doi:10.1038/nm.4377.
9. Concordet, J.-P. & Haeussler, M. CRISPOR: intuitive guide selection for CRISPR/Cas9 genome editing experiments and screens. Nucleic Acids Res. 46, W242-W245 (2018).
10. Listgarten, J. et al. Prediction of off-target activities for the end-to-end design of CRISPR guide RNAs. Nat Biomed Eng 2, 38-47 (2018).
11. Labun, K. et al. CHOPCHOP v3: expanding the CRISPR web toolbox beyond genome editing. Nucleic Acids Res. 47, W171-W174 (2019).
12. Park, J., Bae, S. & Kim, J.-S. Cas-Designer: a web-based tool for choice of CRISPR-Cas9 target sites. Bioinformatics 31, 4014-4016 (2015).
13. Cancellieri, S., Canver, M. C., Bombieri, N., Giugno, R. & Pinello, L. CRISPRitz: rapid, high-throughput and variant-aware in silico off-target site identification for CRISPR genome editing. Bioinformatics 1-8 (2019).
14. Lowy-Gallego, E. et al. Variant calling on the GRCh38 assembly with the data from phase three of the 1000 Genomes Project. Wellcome Open Res 4, 50 (2019).
15. Bergström, A. et al. Insights into human genetic variation and population history from 929 diverse genomes. Science 367, (2020).
16. Karczewski, K. J. et al. The mutational constraint spectrum quantified from variation in 141,456 humans. Nature 581, 434-443 (2020).
17. Canver, M. C. et al. BCL11A enhancer dissection by Cas9-mediated in situ saturating mutagenesis. Nature 527, 192-197 (2015).
18. Wu, Y. et al. Highly efficient therapeutic gene editing of human hematopoietic stem cells. Nature Medicine vol. 25 776-783 (2019).
19. Frangoul, H. et al. CRISPR-Cas9 Gene Editing for Sickle Cell Disease and β-Thalassemia. N. Engl. J. Med. (2020) doi:10.1056/NEJMoa2031054.
20. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
21. ENCODE Project Consortium et al. Expanded encyclopedias of DNA elements in the human and mouse genomes. Nature 583, 699-710 (2020).
22. Demirci, S. et al. Durable and robust fetal globin induction without Anemia in rhesus monkeys following autologous hematopoietic stem cell transplant with BCL11A Erythroid enhancer editing. (2019).
23. Vakulskas, C. A. et al. A high-fidelity Cas9 mutant delivered as a ribonucleoprotein complex enables efficient gene editing in human hematopoietic stem and progenitor cells.
24. Frankish, A. et al. GENCODE reference annotation for the human and mouse genomes. Nucleic Acids Res. 47, D766-D773 (2019).
25. 1000 Genomes Project Consortium et al. A global reference for human genetic variation. Nature 526, 68-74 (2015).
26. McKenna, A. et al. The Genome Analysis Toolkit: a MapReduce framework for analyzing next-generation DNA sequencing data. Genome Res. 20, 1297-1303 (2010).
27. Bolger, A. M., Lohse, M. & Usadel, B. Trimmomatic: a flexible trimmer for Illumina sequence data. Bioinformatics 30, 2114-2120 (2014).
28. Clement, K. et al. CRISPResso2 provides accurate and rapid genome editing sequence analysis. Nat. Biotechnol. 37, 224-226 (2019).

1. A method for enhancing levels of a fetal hemoglobin isoform in a subject in need thereof, the method comprising:
(i) receiving results of a SNP polymorphism assay from a biological sample obtained from a subject indicating the absence of a SNP polymorphism at site rs114518452,and
(ii) administering at least one hematopoietic stem cell that has been modified to inhibit BCL11A to the subject in need thereof, thereby enhancing levels of a fetal hemoglobin isoform in the subject.

2. (canceled)

3. The method of claim 1, wherein the fetal hemoglobin isoform comprises a γ subunit.

4. The method of claim 1, wherein the subject in need thereof comprises a hemoglobinopathy.

5. The method of claim 4, wherein the hemoglobinopathy comprises a sickle cell disease.

6. The method of claim 5, wherein the sickle cell disease is sickle cell anemia, sickle-hemoglobin C disease (HbSC), sickle beta-plus-thalassaemia (HbS/β+), or sickle beta-zero-thalassaemia (HbS/β0).

7. (canceled)

8. The method of claim 1, further comprising a step of analyzing the subject's genome for a SNP polymorphism at site rs114518452.

9. The method of claim 1, wherein the step of modifying the genome comprises contacting the hematopoietic stem cell with a CRISPR/Cas genome modification system and a BCL11A specific guide RNA.

10. The method of claim 9, wherein the BCL11A specific guide RNA comprises BCL11A sgRNA 1617 having a sequence of: CTAACAGTTGCTITTATCAC (SEQ ID NO: 1).

11. The method of claim 1, wherein the hematopoietic stem cell is isolated from the subject and step (ii) is performed ex vivo.

12. The method of claim 1, wherein hematopoietic stem cell is isolated from a donor and step (ii) is performed ex vivo.

13. The method of claim 1, wherein hematopoietic stem cell is derived from an induced pluripotent stem cell or an embryonic stem cell.

14. The method of claim 1, wherein the nucleotide base at site rs114518452 is guanine (G) on the 5′ to 3′ strand.

15. A method for selecting a guide RNA for a CRISPR/Cas based hemoglobinopathy treatment for a subject in need thereof, the method comprising:
(i) receiving results of a SNP polymorphism assay from a biological sample obtained from a subject indicating the absence of a SNP polymorphism at site rs114518452,
(ii) selecting BCL11A sgRNA 1617 as a guide RNA for use with a CRISPR/Cas based hemoglobinopathy treatment.

16. The method of claim 15, wherein the nucleotide base at site rs114518452 is guanine (G) on the 5′ to 3′ strand.

17. The method of claim 15, further comprising a step of analyzing the subject's genome for a SNP polymorphism at site rs114518452.

18. The method of claim 15, further comprising
excluding BCL11A sgRNA 1617 as a guide RNA for use with a CRISPR/Cas based hemoglobinopathy treatment.

19. The method of claim 18, wherein the nucleotide base at site rs114518452 is cytosine (C) on the 5′ to 3′ strand.

20. The method of claim 18, further comprising a step of analyzing the subject's genome for a SNP polymorphism at site rs114518452.

21.-23. (canceled)

24. A hematopoietic stem cell composition comprising a hematopoietic stem cell modified to inhibit BCL11A for use in a method of treating a subject with a hemoglobinopathy, wherein the method comprises receiving results of a SNP polymorphism assay from a biological sample obtained from the subject indicating the absence of a SNP polymorphism at site rs114518452.
